(19) United States
(12) Patent Application Publication
(10) Pub. No.: US 2002/0068702 A1
(43) Pub. Date: Jun. 6, 2002

(54) Novel peptides as NS3-serine protease inhibitors of hepatitis C virus

(76) Inventors: Marguerita Lim-Wilby, La Jolla, CA (US); Odile E. Levy, San Diego, CA (US); Terrence K. Brunck, Santa Fe, NM (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 09/909,164

(22) Filed: Jul. 19, 2001

Related U.S. Application Data
(60) Provisional application No. 60/220,101, filed on Jul. 21, 2000.

(51) Int. Cl. A61K 38/00; C07K 5/00; C07K 7/00; C07K 16/00; C07K 17/00; A61K 38/04; A61K 9/20

(52) U.S. Cl. 514/16; 530/327; 424/464

(57) ABSTRACT

The present invention discloses novel peptide compounds containing eleven amino acid residues which have HCV protease inhibitory activity as well as methods for preparing such compounds. In another embodiment, the invention discloses pharmaceutical compositions comprising such peptides as well as methods of using them to treat disorders associated with the HCV protease.

### FIELD OF INVENTION

The present invention relates to novel hepatitis C virus (“HCV”) protease inhibitors, pharmaceutical compositions containing one or more such inhibitors, methods of preparing such inhibitors and methods of using such inhibitors to treat hepatitis C and related disorders. This invention specifically discloses novel peptide compounds containing eleven amino acid residues as inhibitors of the HCV NS3/NS4a serine protease.

### BACKGROUND OF THE INVENTION

Hepatitis C virus (HCV) is a (+)-sense single-stranded RNA virus that has been implicated as the major causative agent in non-A, non-B hepatitis (NANBH), particularly in blood-associated NANBH (BB-NANBH)(see, International Patent Application Publication No. WO 89/04669 and European Patent Application Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

Recently, an HCV protease necessary for polypeptide processing and viral replication has been identified, cloned and expressed; (see, e.g., U.S. Pat. No. 5,712,145). This approximately 3000 amino acid polyprotein contains, from the amino terminus to the carboxy terminus, a nucleocapsid protein (C), envelope proteins (E1 and E2) and several non-structural proteins (NS1, 2, 3, 4a, 5a and 5b). NS3 is an approximately 68 kda protein, encoded by approximately 1893 nucleotides of the HCV genome, and has two distinct domains: (a) a serine protease domain consisting of approximately 200 of the N-terminal amino acids; and (b) an RNA-dependent ATPase domain at the C-terminus of the protein. The NS3 protease is considered a member of the chymotrypsin family because of similarities in protein sequence, overall three-dimensional structure and mechanism of catalysis. Other chymotrypsin-like enzymes are elastase, factor Xa, thrombin, trypsin, plasmin, urokinase, tPA and PSA. The HCV NS3 serine protease is responsible for proteolysis of the polypeptide (polyprotein) at the NS3/NS4a, NS4a/NS4b, NS4b/NS5a and NS5a/NS5b junctions and is thus responsible for generating four viral proteins during viral replication. This has made the HCV NS3 serine protease an attractive target for antiviral chemotherapy.

It has been determined that the NS4a protein, an approximately 6 kda polypeptide, is a co-factor for the serine protease activity of NS3. Autocleavage of the NS3/NS4a junction by the NS3/NS4a serine protease occurs intramolecularly (i.e., cis) while the other cleavage sites are processed intermolecularly (i.e., trans).

Analysis of the natural cleavage sites for HCV protease revealed the presence of cysteine at P1 and serine at P1′ and that these residues are strictly conserved in the NS4a/NS4b, NS4b/NS5a and NS5a/NS5b junctions. The NS3/NS4a junction contains a threonine at P1 and a serine at P1′. The Cys→Thr substitution at NS3/NS4a is postulated to account for the requirement of cis rather than trans processing at this junction. See, e.g., Pizzi et al. (1994) Proc. Natl. Acad. Sci (USA) 91:888-892, Failla et al. (1996) Folding & Design 1:35-42. The NS3/NS4a cleavage site is also more tolerant of mutagenesis than the other sites. See, e.g., Kollykhalov et al. (1994) J. Virol. 68:7525-7533. It has also been found that acidic residues in the region upstream of the cleavage site are required for efficient cleavage. See, e.g., Komoda et al. (1994) J. Virol. 68:7351-7357.

Inhibitors of HCV protease that have been reported include antioxidants (see, International Patent Application Publication No. WO 98/14181), certain peptides and peptide analogs (see, International Patent Application Publication No. WO 98/17679, Landro et al. (1997) Biochem. 36:9340-9348, Ingallinella et al. (1998) Biochem. 37:8906-8914, Llinàs-Brunet et al. (1998) Bioorg. Med. Chem. Lett. 8:1713-1718), inhibitors based on the 70-amino acid polypeptide eglin c (Martin et al. (1998) Biochem. 37:11459-11468, inhibitors affinity selected from human pancreatic secretory trypsin inhibitor (hPSTI-C3) and minibody repertoires (MBip) (Dimasi et al. (1997) J. Virol. 71:7461-7469), cV_{H}E2 (a “camelized” variable domain antibody fragment) (Martin et al.(1997) Protein Eng. 10:607-614), and α1-antichymotrypsin (ACT) (Elzouki et al.) (1997) J. Hepat. 27:42-28). A ribozyme designed to selectively destroy hepatitis C virus RNA has recently been disclosed (see, BioWorld Today 9(217): 4 (Nov. 10, 1998)).

Reference is also made to the PCT Publications, No. WO 98/17679, published Apr. 30, 1998 (Vertex Pharmaceuticals Incorporated); WO 98/22496, published May 28,1998 (F. Hoffmann-La Roche AG); and WO 99/07734, published Feb. 18, 1999 (Boehringer Ingelheim Canada Ltd.).

HCV has been implicated in cirrhosis of the liver and in induction of hepatocellular carcinoma. The prognosis for patients suffering from HCV infection is currently poor. HCV infection is more difficult to treat than other forms of hepatitis due to the lack of immunity or remission associated with HCV infection. Current data indicates a less than 50% survival rate at four years post cirrhosis diagnosis. Patients diagnosed with localized resectable hepatocellular carcinoma have a five-year survival rate of 10-30%, whereas those with localized unresectable hepatocellular carcinoma have a five-year survival rate of less than 1%.

Reference is made to A. Marchetti et al, Synlett, S1, 1000-1002 (1999) describing the synthesis of bicylic analogs of an inhibitor of HCV NS3 protease. A compound disclosed therein has the formula:

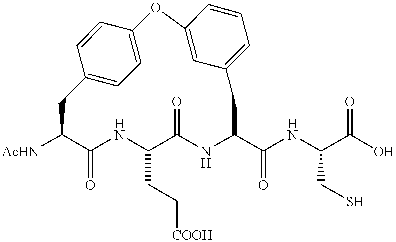

Reference is also made to WO 00/09558 (Assignee: Boehringer Ingelheim Limited; Published Feb. 24, 2000) which discloses peptide derivatives of the formula:

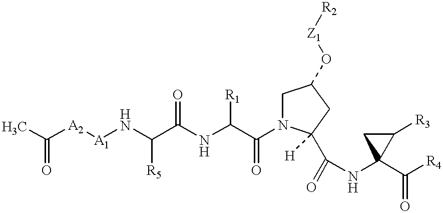

where the various elements are defined therein. An illustrative compound of that series is:

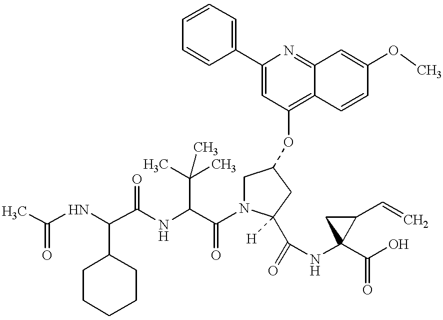

Reference is also made to WO 00/09543 (Assignee: Boehringer Ingelheim Limited; Published Feb. 24, 2000) which discloses peptide derivatives of the formula:

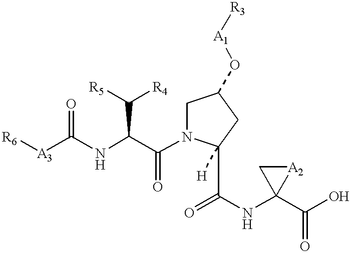

where the various elements are defined therein. An illustrative compound of that series is:

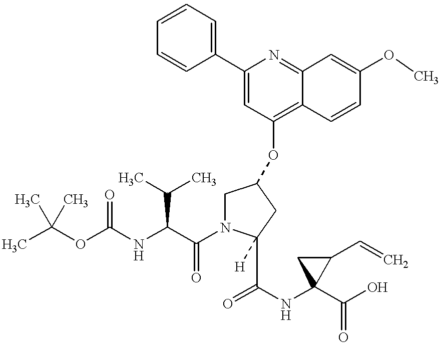

Current therapies for hepatitis C include interferon-α (INF_{α}) and combination therapy with ribavirin and interferon. See, e.g., Beremguer et al. (1998) Proc. Assoc. Am. Physicians 110(2):98-112. These therapies suffer from a low sustained response rate and frequent side effects. See, e.g., Hoofnagle et al. (1997) N. Engl. J. Med. 336:347. Currently, no vaccine is available for HCV infection.

Pending and copending U.S. patent applications, Ser. No. ______, filed ______, and Ser. No. ______, filed ______, Ser. No. ______, filed ______, Ser. No. ______, filed ______, Ser. No. ______, filed ______, and Ser. No. ______, filed ______, disclose various types of peptides as NS-3 serine protease inhibitors of hepatitis C virus.

There is a need for new treatments and therapies for HCV infection. It is, therefore, an object of this invention to provide compounds useful in the treatment or prevention or amelioration of one or more symptoms of hepatitis C.

It is a further object herein to provide methods of treatment or prevention or amelioration of one or more symptoms of hepatitis C.

A still further object of the present invention is to provide methods for modulating the activity of serine proteases, particularly the HCV NS3/NS4a serine protease, using the compounds provided herein.

Another object herein is to provide methods of modulating the processing of the HCV polypeptide using the compounds provided herein.

### SUMMARY OF THE INVENTION

In its many embodiments, the present invention provides a novel class of inhibitors of the HCV protease, pharmaceutical compositions containing one or more of the compounds, methods of preparing pharmaceutical formulations comprising one or more such compounds, and methods of treatment, prevention or amelioration or one or more of the symptoms of hepatitis C. Also provided are methods of modulating the interaction of an HCV polypeptide with HCV protease. Among the compounds provided herein, compounds that inhibit HCV NS3/NS4a serine protease activity are preferred. The presently disclosed compounds generally contain eleven amino acid residues. The compounds are α-ketoamide peptide analogs. The compounds generally contain eleven amino acid residues. There is a α-ketoamide group at the P1 position of the compounds. The compounds are capped at the N-terminus with an acyl, carbamoyl or sulfonyl group and are C-terminal amides, esters and acids.

In one embodiment, the compounds have Formula I:

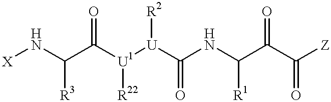

or a pharmaceutically acceptable derivative thereof, where X is: COCH(R⁴)NHCO—CH(R⁵)NHCOCH(R⁶)NHCORⁿ or COCH(R⁴)NHCOCH(R⁵)NHCOCH(R⁶)—NHSO₂R²⁰;

U¹ is a nitrogen atom and U is —CH—;

Z is: NH—CH(R^{1′})CONHCH(R^{2′})CONHCH(R^{3′})CONHCH(R^{4′})CONHCH(R^{5′})COR^{c};

R¹, R², R²², R³, R⁴, R⁵, R⁶, Rⁿ, R^{2′}, R^{3′}, R^{4′}, R^{5′}, R^{1′}, R²⁰, and R^{c} are selected from (a) and (b) as follows:

(a) R¹ is selected from (i)-(v) as follows:

(i) C₁₋₂ alkyl substituted with Q;

(ii) C₃₋₁₀ alkyl that is unsubstituted or substituted with Q;

(iii) cycloalkyl that is unsubstituted or substituted with Q;

(iv) alkenyl that is unsubstituted or substituted with Q; or

(v) alkynyl that is unsubstituted or substituted with Q;

R² and R²² are selected from (i) or (ii) as follows:

(i) R² and R²² together form alkylene, alkenylene, thiaalkylene, thiaalkenylene, alkylenethiaalkylene, alkyleneazaalkylene, arylene, alkylenearylene or dialkylenearylene; or

(ii) R² and R²² are each independently selected from H, alkyl, cycloalkyl, aralkyl and heteroaralkyl;

R³ is selected from the group consisting of alkyl, cycloalkyl, aryl, aralkyl, heteroaryl and heteroaralkyl;

R⁴ is alkyl, cycloalkyl, heteroaralkyl or aralkyl;

R⁵ is alkyl or cycloalkyl;

R⁶ is alkyl or cycloalkyl;

Rⁿ is alkyl, alkenyl, alkynyl, alkoxy, aryl, aralkyl, aralkenyl, aralkynyl, aryloxy, aralkoxy, heteroaryl, heteroaralkyl, heteroaralkenyl, heteroaralkynyl, heteroaryloxy, heteroaralkoxy or NR³⁰R³¹;

R³⁰ and R³¹ are each independently selected from the group consisting of H, alkyl, aryl, heteroaryl, aralkyl and heteroaralkyl;

R^{2′} is H, alkyl, cycloalkyl, aryl, heteroaryl, aralkyl or heteroaralkyl;

R^{3′} is selected from the group consisting of alkyl, cycloalkyl, aralkyl and heteroaralkyl;

R^{4′} is aralkyl or heteroaralkyl;

R^{5′} is alkyl or cycloalkyl;

R^{1′} is selected from H, alkyl, cycloalkyl, aralkyl and heteroaralkyl;

R²⁰ is alkyl, alkenyl, alkynyl, aryl, aralkyl, aralkenyl, aralkynyl, heteroaryl, heteroaralkyl, heteroaralkenyl or heteroaralkynyl;

R^{c} is selected from amino, hydroxy, alkoxy, cycloalkoxy, alkylamino, alkenyloxy, alkenylamino, aryloxy, heteroaryloxy, arylamino, heteroarylamino, aralkoxy, heteroaralkoxy, aralkylamino and heteroaralkyl-amino;

Q is halide, pseudohalide, hydroxy, nitrile, formyl, mercapto, alkyl, haloalkyl, polyhaloalkyl, alkenyl containing 1 double bond, alkynyl containing 1 triple bond, cycloalkyl, cycloalkylalkyl, alkylidene, alkylcarbonyl, alkoxy, perfluoroalkoxy, alkylcarbonyloxy or alkylthio; and

R², R²², R³, R⁴, R⁵, R⁶, Rⁿ, R^{2′}, R^{3′}, R^{4′}, R^{5′}, R^{1′}, R²⁰, and R^{c} are unsubstituted or substituted with one or more substituents each independently selected from Q¹, where Q¹ is halide, pseudohalide, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, ureido, alkylureido, arylureido, amino, aminoalkyl, alkylaminoalkyl, dialkylaminoalkyl, arylaminoalkyl, diarylaminoalkyl, alkylarylaminoalkyl, alkylamino, dialkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxy-arylcarbonylamino, aryloxycarbonylamino, alkylsulfonylamino, arylsulfonylamino, azido, dialkylphosphonyl, alkylarylphosphonyl, diarylphosphonyl, alkylthio, arylthio, perfluoroalkylthio, hydroxycarbonylalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl, arylaminosulfonyl, diarylaminosulfonyl or alkylarylaminosulfonyl; and

the aryl and heteroaryl groups of Q¹ are unsubstituted or substituted with one or more substituents each independently selected from Q², where Q² is alkyl, halide, pseudohalide, alkoxy, aryloxy or alkylenedioxy; or

(b) R¹ and R³, and/or R² and R⁴, and/or R³ and R⁵, and/or R⁴ and R⁶, and/or R¹ and R^{2′}, and/or R^{1′} and R^{3′}, and/or R^{2′} and R^{4′}, and/or R^{3′} and R^{5′}, and/or R² and R^{1′}, and/or R¹ and R^{1′} together form alkylene, alkenylene, alkylenearylene, dialkylenearylene, alkylene-OC(O)-alkylene, alkylene-NHC(O)-alkylene, alkylene-O-alkylene, alkylene-NHC(O)-alkylene-NHC(O)-alkylene, alkylene-C(O)NH-alkylene-NHC(O)-alkylene, alkylene-NHC(O)-alkylene-C(O)NH-alkylene, alkylene-S(O)ₘ—S(O)ₘ-alkylene or alkylene-S(O)ₘ-alkylene where m is 0-2, and the alkylene and arylene portions are unsubstituted or substituted with Q¹; and the others are chosen as in (a).

In more preferred embodiments, the compounds are chosen with the proviso that if R² and R²² together form unsubstituted propylene, then R¹ is not i-Pr, i-Bu or 2-(methylthio)ethyl.

In other preferred embodiments, Q is halide, pseudohalide, hydroxy, nitrile, formyl, mercapto, alkyl, haloalkyl, polyhaloalkyl, alkenyl containing 1 double bond, alkynyl containing 1 triple bond, cycloalkyl, cycloalkylalkyl, alkylidene, alkylcarbonyl, alkoxy, perfluoroalkoxy, alkylcarbonyloxy or alkylthio.

In all embodiments described herein, R¹ is preferably C₃₋₁₀ alkyl, or is alkenyl or alkynyl, and is unsubstituted or is substituted with Q. In preferred embodiments, R¹ is n-Pr, allyl or propynyl, most preferably n-Pr.

In other preferred embodiments, U is —CH— and U¹ is a nitrogen atom.

The P1-P6 and P1′-P5′ residues are described in further detail below. It is to be understood that these residues are selected independently of each other to arrive at the compounds provided herein. Thus, any combination of the P1-P6 and P1′-P5′ residues described herein is encompassed within the embodiments provided herein. Preferred combinations of these residues are described in detail herein, and are those that provide compounds with the highest HCV protease, particularly the highest HCV NS3/NS4a serine protease, inhibitory activity and/or desirable pharmacokinetic properties, including but not limited to, oral bioavailability, in vivo half life, etc.

The groups R², R²², R³, R⁴, R⁵, R⁶, Rⁿ, R^{2′}, R^{3′}, R^{4′}, R^{5′}, R^{1′}, R²⁰, and R^{c} described in detail below are unsubstituted or substituted with one or more substituents each independently selected from Q¹, where Q¹ is halide, pseudo-halide, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, ureido, alkylureido, arylureido, amino, aminoalkyl, alkylaminoalkyl, dialkylaminoalkyl, arylaminoalkyl, diarylaminoalkyl, alkylarylaminoalkyl, alkylamino, dialkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, azido, dialkylphosphonyl, alkylarylphosphonyl, diarylphosphonyl, alkylthio, arylthio, perfluoroalkylthio, hydroxycarbonylalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl, arylaminosulfonyl, diarylaminosulfonyl or alkylarylaminosulfonyl; wherein the aryl and heteroaryl groups of Q¹ are unsubstituted or substituted with one or more substituents each independently selected from Q², where Q² is alkyl, halide, pseudohalide, alkoxy, aryloxy or alkylenedioxy.

1. The P1 Residue

In the embodiments described in detail herein, the side chain of the P1 residue (R¹) is selected from (i)-(v) as follows:

(i) C₁₋₂ alkyl substituted with Q;

(ii) C₃₋₁₀ alkyl that is unsubstituted or substituted with Q;

(iii) cycloalkyl that is unsubstituted or substituted with Q;

(iv) alkenyl that is unsubstituted or substituted with Q; and

(v) alkynyl that is unsubstituted or substituted with Q, where Q is halide, pseudohalide, hydroxy, nitrile, formyl, mercapto, alkyl, haloalkyl, polyhaloalkyl, alkenyl containing 1 double bond, alkynyl containing 1 triple bond, cycloalkyl, cycloalkylalkyl, alkylidene, alkylcarbonyl, alkoxy, perfluoroalkoxy, alkylcarbonyloxy or alkylthio.

In preferred embodiments, R¹ is selected from (i)-(iv) as follows:

(i) C₁₋₂ alkyl substituted with Q;

(ii) C₃₋₁₀ alkyl that is unsubstituted or substituted with Q;

(iii) alkenyl that is unsubstituted or substituted with Q; and

(iv) alkynyl that is unsubstituted or substituted with Q.

In more preferred embodiments, R¹ is C₃₋₁₀ alkyl, or is alkenyl or alkynyl, and is unsubstituted or substituted with Q. R¹ is more preferably C₃₋₁₀ alkyl or is alkynyl, most preferably C₃₋₁₀ alkyl.

Thus, in particularly preferred embodiments, R¹ is selected from groups such as n-Pr, CH₂C≡CH, i-Bu, n-Bu, i-Pr, CH₂CH═CH₂, hydroxymethyl, CH₂SH, CH₂CH₂SH, CH₂SMe, 2-(methylthio)ethyl, CH₂SEt, 1-hydroxy-1-ethyl and methoxymethyl. R¹ is more preferably n-Pr, allyl or propynyl, most preferably n-Pr. Thus, the P1 residue is most preferably norvaline.

2. The P2 Residue

In the embodiments described herein, the P2 residue is a cyclic amino acid or amino acid analog, or has a side chain selected from H, alkyl, cycloalkyl, aralkyl and heteroaralkyl. In certain embodiments, the substituents at the P2 residue are selected as follows:

one of U and U¹ is a nitrogen atom and the other is —CH— or —C(lower alkyl)—; and

R² and R²² are selected from (i) or (ii) as follows:

(i) R² and R²² together form alkylene, alkenylene, thiaalkylene, thiaalkenylene, alkylenethiaalkylene, alkyleneazaalkylene, arylene, alkylenearylene or dialkylenearylene; or

(ii) R² and R²² are each independently selected from H, alkyl, cycloalkyl, aralkyl and heteroaralkyl.

In certain preferred embodiments, R² and R²² are selected with the proviso that if R² and R²² together form unsubstituted propylene, then R¹ is not i-Pr, i-Bu or 2-(methylthio)ethyl.

In preferred embodiments, U is —CH— or —C(lower alkyl)— and U¹ is a nitrogen atom. U is more preferably —CH— or —C(Me)—, most preferably —CH—.

In other preferred embodiments, R² and R²² are selected from (i) or (ii) as follows:

(i) R² and R²² together form alkylene, thiaalkylene, or dialkylenearylene; or

(ii) R² and R²² are each independently selected from H, alkyl and aralkyl.

In more preferred embodiments, R² and R²² are selected from (i) or (ii) as follows:

(i) R² and R²² together form propylene, butylene or 1,2-dimethylenephenylene, where the butylene and 1,2-dimethylenephenylene groups are unsubstituted and the propylene group is unsubstituted or is substituted with 4-methoxyphenylsulfonylamino, N-phenylureidomethyl, methyl, benzoylaminomethyl, phenyl, 3-phenoxybenzoylaminomethyl, N-phenylureido, phenylsulfonylaminomethyl, 9-fluorenylmethoxycarbonylaminomethyl, phenoxy-carbonylaminomethyl, iso-butoxycarbonylamino, hydroxycarbonylmethyl, hydroxycarbonylmethoxy, 2-propen-1-yl, N-(4-methoxyphenyl)ureido, 3-phenoxybenzoylamino, 4-methoxyphenylmethyl, 9-fluorenylmethoxycarbonylamino, benzyl, 4-methoxybenzoylamino, benzoylamino, 3,4-methylened ioxybenzoylamino, 4-fluorobenzoylamino, phenylsulfonylamino, 4-phenoxybenzoylamino or amino; or

(ii) R² is selected from CH₂SO₂Me, CH₂SCH₂COOH, CH₂CH₂COOH and CH₂SMe; and R²² is H.

In particularly preferred embodiments, R² and R²² are selected from (i) or (ii) as follows:

(i) R² and R²² together form propylene or 1,2-dimethylenephenylene, where the 1,2-dimethylenephenylene group is unsubstituted and the propylene group is unsubstituted or is substituted with 4-methoxyphenylsulfonylamino, N-phenylureidomethyl, methyl, benzoylaminomethyl, phenyl, 3-phen-oxybenzoylaminomethyl, N-phenylureido, phenylsulfonylaminomethyl, 9-fluorenyl-methoxycarbonylaminomethyl, phenoxycarbonylaminomethyl, iso-butoxy-carbonylamino, hydroxycarbonylmethyl or hydroxycarbonylmethoxy; or

(ii) R² is selected from CH₂SO₂Me and CH₂SCH₂COOH; and R²² is H.

In particularly preferred embodiments, the P2 residue is a cyclic amino acid analog, preferably a substituted proline. In these embodiments, R² and R²² together form propylene or 1,2-dimethylenephenylene, where the 1,2-dimethylenephenylene group is unsubstituted and the propylene group is unsubstituted or is substituted with 4-methoxyphenylsulfonylamino, N-phenyl-ureidomethyl, methyl, benzoylaminomethyl, phenyl, 3-phen-oxybenzoylaminomethyl, N-phenylureido, phenylsulfonylaminomethyl, 9-fluorenyl-methoxycarbonylaminomethyl, phenoxycarbonylaminomethyl, iso-butoxy-carbonylamino, hydroxycarbonylmethyl or hydroxycarbonylmethoxy. R² and R²² most preferably together form unsubstituted propylene.

In other embodiments, R² and R²² are selected from (i) or (ii) as follows:

(i) R² is CH₂R⁴⁰, CH₂CH₂R⁴⁰, CH₂CH₂NH-R⁴⁰ or CH₂-(4-hydroxy-3-R⁴⁰-phenyl), and R²² is H, alkyl, cycloalkyl, aralkyl or heteroaralkyl; or

(ii) R² and R²² together form —CH₂CH(R⁴⁰)CH₂— or

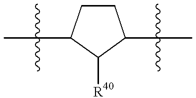

where R⁴⁰ is (L¹)ₛ-R³²-L²-R³³ in which L¹ is selected from (CH₂)_{z}NHC(O), (CH₂)_{z}OC(O), (CH₂)_{z}OC(O)NH, O(CH₂)_{z}C(O), SO₂, C(O) and (CH₂)_{z}, where z is 0 to 3; s is 0 or 1; R³² is 1,3-phenylene, 4-hydroxy-1,3-phenylene, 2,4-pyridylene, 5,7-indolylene, or

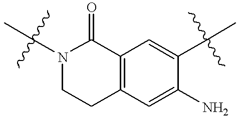

L² is O or CH₂; R³³ is 4,6-dimethoxy-2,3-methylenedioxyphenyl, naphthyl, x or

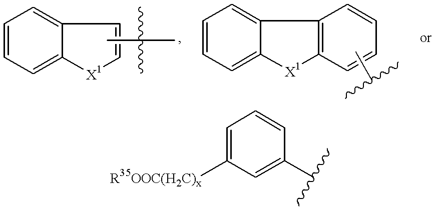

x is 0-4; R³⁵ is H or alkyl; and X¹ is NR³⁶, O, S or CH₂, where R³⁶ is H, alkyl, aryl or heteroaryl.

In other preferred embodiments, R² and R²² together form —CH₂C(R⁴⁰)(R⁴¹)CH₂— or

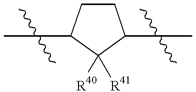

where R⁴⁰ and R⁴¹ are selected from (i), (ii) and (iii) as follows:

(i) R⁴⁰ is (L¹)ₛ—R³²—L²-R³³; and R⁴¹ is selected from H, alkyl, alkenyl, alkynyl and cycloalkyl; or

(ii) R⁴⁰ and R⁴¹ are each independently selected from —S-alkyl, —S-aryl, —S-aralkyl, —O-alkyl, —O-aryl and —O-aralkyl; or

(iii) R⁴⁰ and R⁴¹ together form —S-alkylene-S—, —S-alkylene-O—, —O-alkylene—O—, —S-arylene-S—, —O-arylene-O— or —O-arylene-S—;

L¹ is selected from (CH₂)_{z}NHC(O), (CH₂)_{z}OC(O), (CH₂)_{z}OC(O)NH, O(CH₂)_{z}C(O), SO₂, C(O) and (CH₂)_{z}, where z is 0 to 3;

s is 0 or 1;

R³² is 1,3-phenylene, 4-hydroxy-1,3-phenylene, 2,4-pyridylene, 5,7-indolylene, or

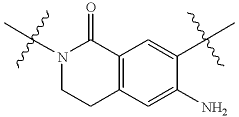

L² is O or CH₂;

R³³ is 4,6-dimethoxy-2,3-methylenedioxyphenyl, naphthyl,

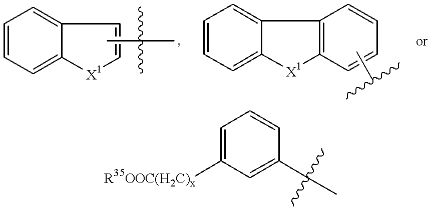

where x is 0-4, R³⁵ is H or alkyl, and X¹ is NR³⁶, O, S or CH₂, where R³⁶ is H, alkyl, aryl or heteroaryl.

In other embodiments, R² and R²² together form —C(R⁵⁰)(R⁵¹)—C(R⁵²)(R⁵³)—CH₂—, where R⁵⁰ and R⁵¹ are attached to the carbon adjacent to U and each are independently hydrogen or lower alkyl; R⁵² is cis to the carbonyl group attached to U and is hydrogen or hydroxy; and R⁵³ is trans to the carbonyl group attached to U and is —(CH₂)_{z}-phenyl, ethynylphenyl, ethenylphenyl, alkenyl, alkynyl, —(CH₂)_{z}-aminocarbonylphenyl, —(CH₂)_{z}—aminosulfonylphenyl, —(CH₂)_{z}-aminocarbonyloxyphenyl or —(CH₂)_{z}—COOH, where z is 0-3 and the phenyl portions of R⁵³ are unsubstituted or substituted with one or more substitutents independently selected from Q⁴, wherein Q⁴ is alkoxy, halide, pseudohalide, aryloxy or alkylenedioxy.

In certain embodiments herein, the P2 residue is a 4-trans-substituted proline derivative. In these embodiments, R² and R²² form propylene that is substituted at the 2-position of the propylene chain.

3. The P3 and P4 Residues

In the embodiments described herein, the P3 and P4 residues are hydrophobic amino acid residues or analogs thereof. Thus, in these embodiments, R³ and R⁴ are selected from alkyl, cycloalkyl, aryl, aralkyl, heteroaryl and heteroaralkyl.

R³ is preferably alkyl, cycloalkyl, aryl or aralkyl, more preferably alkyl or cycloalkyl, particularly isopropyl, 1-methyl-1-propyl or cyclohexyl, most preferably isopropyl or cyclohexyl. Preferred P3 residues are valine, isoleucine and cyclohexylglycine, most preferred are valine or cyclohexylglycine.

R⁴ is preferably alkyl, cycloalkyl, heteroaralkyl or aralkyl, more preferably alkyl, heteroaralkyl or aralkyl, particularly alkyl, most preferably isopropyl. Thus, the most preferred P4 residue is valine.

In other embodiments, the P3 and/or P4 residues are amino acid residues or analogs thereof that induce a β-strand. In these embodiments, R³ and/or R⁴ is CH(R²⁵)(R²⁶) or cycloalkyl; R²⁵ and R²⁶ are each independently selected from alkyl, alkenyl, alkynyl, aryl, heteroaryl, aralkyl and heteroaralkyl; and R²⁵, R²⁶ and cycloalkyl are unsubstituted or substituted with Q¹. In certain embodiments, R³ and/or R⁴ is the side chain of valine, isoleucine or cyclohexylglycine.

4. The P5 and P6 Residues

In the embodiments described in detail herein, the P5 and P6 are residues that possess acidic side chains. Thus, R⁵ and R⁶ are each independently alkyl or cycloalkyl that is substituted with an acidic group including, but not limited to, carboxy. In preferred embodiments, R⁵ and R⁶ are each independently (CH₂)ₜCOOH, where t is 1-6, preferably 1-4, more preferably 2. Thus, R⁵ and R⁶ are each preferably CH₂COOH or CH₂CH₂COOH, more preferably CH₂CH₂COOH. Preferred residues at P5 and P6 are aspartic or glutamic acid, most preferred is glutamic acid.

5. The P1′ Residue

In the embodiments described in detail herein, the compounds provided herein preferably contain an amino acid residue or analog thereof at the P1′ position. In these embodiments, R¹ is selected from hydrogen, alkyl, cycloalkyl, aralkyl and heteroaralkyl. In more preferred embodiments, R¹ is hydrogen, alkyl or aralkyl, most preferably hydrogen. Thus, the P1′ residue is preferably glycine.

6. The P2′-P5′ Residues

The compounds described herein may possess amino acid residues or analogs thereof at the P2′-P5′ positions. In these embodiments, the substituents R²-R⁵ are each independently selected from hydrogen, alkyl, cycloalkyl, aryl, heteroaryl, aralkyl and heteroaralkyl. More preferred substituents are each independently selected from hydrogen, alkyl, aralkyl and heteroaralkyl. Particularly preferred groups for each substituent R²′-R⁵ are described in detail below.

R²′ is preferably hydrogen, alkyl, cycloalkyl, aryl or heteroaryl; more preferably hydrogen or alkyl; most preferably CH₂CH₂SMe, C(OH)Me, CH₂CH₂S(O)Me or CH₂C(O)NH₂. Thus, the most preferred P2′ residues are methionine, threonine, the sulfoxide of methionine, and asparagine.

R^{3′} is preferably alkyl, cycloalkyl, aralkyl or heteroaralkyl; more preferably alkyl or heteroaralkyl; most preferably hydroxymethyl, hydroxycarbonylmethyl or 4-imidazolylmethyl. Thus, the most preferred P3′ residues are serine, aspartic acid and histidine.

R^{4′} is preferably aralkyl or heteroaralkyl; more preferably aralkyl; most preferably 4-hydroxyphenylmethyl. Thus, the most preferred P4′ residue is tyrosine.

R^{5′} is preferably alkyl or cycloalkyl; more preferably alkyl; most preferably hydroxymethyl. Thus, the most preferred P5′ residue is serine.

In embodiments described in detail herein, the C-terminal group, Z is NH—CH(R^{1′})CONHCH(R^{2′})CONHCH(R^{3′})CONHCH(R^{4′})CONHCH(R^{5′})COR^{c}, where R^{1′}-R^{5′} are selected as described above and R^{c} is selected from amino, hydroxy, alkoxy, cycloalkoxy, alkylamino, alkenyloxy, alkenylamino, aryloxy, heteroaryloxy, arylamino, heteroarylamino, aralkylamino and heteroaralkylamino. R^{c} is preferably hydroxy, alkoxy or amino, more preferably OH, OEt, NH₂ or O-allyl; particularly OH, OEt or NH₂; most preferably OH or NH₂.

In all embodiments described herein, at least one of X and Z is an amino acid residue or analog thereof and the compounds provided herein contain eleven amino acid residues or analogs thereof.

7. The X Group

In preferred embodiments, X is: COCH(R⁴)NHCOCH(R⁵)NHCOCH(R⁶)—NHCORⁿ or COCH(R⁴)NHCOCH(R⁵)NHCOCH(R⁶)NHSO₂R²⁰; where R⁴-R⁶, Rⁿ and R²⁰ are selected as described above.

In all embodiments described herein, at least one of X and Z is an amino acid residue or analog thereof and the compounds provided herein contain from four up to eleven amino acid residues or analogs thereof.

In more preferred embodiments, Rⁿ is alkyl, alkoxy, heteroaryl, aryl or aralkyl; more preferably alkyl, aryl or heteroaryl; particularly alkyl; most preferably methyl. In other more preferred embodiments, R²⁰ is alkyl, aralkyl, aryl or aralkenyl; preferably methyl, camphoryl, benzyl, phenyl or styryl.

8. Some other Preferred Embodiments

As noted above, the side chain groups of the P1-P6 and P1′-P5′ residues (i.e., R¹-R⁶ and R^{1′}-R^{5′}) are selected as described above and are selected independently of each other to arrive at the compounds provided herein. Thus, any combination of the P1-P6 and P1′-P5′ residues described herein is encompassed within the embodiments provided herein. Preferred combinations of these residues are described in detail below.

In preferred embodiments herein, the residues at the P1-P3 positions of the compounds (i.e., the R¹, R², R²² and R³ substituents) are chosen to provide compounds that have the highest HCV protease, preferably the highest HCV NS3/NS4a serine protease, activity. More preferred residues are those described in detail below, or may be determined using assays known to those of skill in the art, such as the assays exemplified herein.

In certain embodiments, the compounds have formula I, where R¹ is C₃₋₁₀ alkyl, or is alkenyl or alkynyl, preferably C₃₋₁₀ alkyl or alkynyl, more preferably C₃₋₁₀ alkyl, most preferably n-Pr, and is unsubstituted or substituted with Q;

R² and R²² are selected from (i) or (ii) as follows:

(i) R² and R²² together form propylene, butylene or 1,2-dimethylenephenylene, where the butylene and 1,2-dimethylenephenylene groups are unsubstituted and the propylene group is unsubstituted or is substituted with 4-methoxyphenylsulfonylamino, N-phenylureidomethyl, methyl, benzoylaminomethyl, phenyl, 3-phenoxybenzoylaminomethyl, N-phenylureido, phenylsulfonylaminomethyl, 9-fluorenylmethoxy-carbonylaminomethyl, phenoxycarbonylaminomethyl, iso-butoxy-carbonylamino, hydroxycarbonylmethyl, hydroxycarbonylmethoxy, 2-propen-1-yl, N-(4-methoxyphenyl)ureido, 3-phenoxybenzoylamino, 4-methoxyphenylmethyl, 9-fluorenylmethoxycarbonylamino, benzyl, 4-methoxybenzoylamino, benzoylamino, 3,4-methylenedioxybenzoylamino, 4-fluorobenzoylamino, phenylsulfonylamino, 4-phenoxybenzoylamino or amino; or

(ii) R² is selected from CH₂SO₂Me, CH₂SCH₂COOH, CH₂CH₂COOH and CH₂SMe, preferably from CH₂SO₂Me and CH₂SCH₂COOH; and R²² is H;

R³ is i-Pr, cyclohexyl or 1-methyl-1-propyl; and U, U¹, X and Z are as described above.

In other embodiments, the compounds have formula I, where R¹ is C₃₋₁₀ alkyl, or is alkenyl or alkynyl, preferably C₃₋₁₀ alkyl or alkynyl, more preferably C₃₋₁₀ alkyl, most preferably n-Pr, and is unsubstituted or substituted with Q;

R² and R²² are selected from (i) or (ii) as follows:

(i) R² and R²² together form propylene or 1,2-dimethylenephenylene, where the 1,2-dimethylenephenylene group is unsubstituted and the propylene group is unsubstituted or is subsituted with 4-methoxyphenylsulfonylamino, N-phenylureidomethyl, methyl, benzoylaminomethyl, phenyl, 3-phenoxybenzoylaminomethyl, N-phenylureido, phenylsulfonylaminomethyl, 9-fluorenylmethoxycarbonylaminomethyl, phenoxycarbonylaminomethyl, iso-butoxycarbonylamino, hydroxycarbonylmethyl or hydroxycarbonylmethoxy; or

(ii) R² is selected from CH₂SO₂Me and CH₂SCH₂COOH; and R²² is H;

R³ is i-Pr, cyclohexyl or 1-methyl-1-propyl; and U, U¹, X and Z are as described above.

In other preferred embodiments, the compounds have formula I, where R¹ is C₃₋₁₀ alkyl, or is alkenyl or alkynyl, preferably n-Pr, allyl or propynyl, more preferably n-Pr or propynyl, most preferably n-Pr, and is unsubstituted or substituted with Q;

R² and R²² are selected from (i) or (ii) as follows:

(i) R² and R²² together form propylene, butylene or 1,2-dimethylenephenylene, where the butylene and 1,2-dimethylenephenylene groups are unsubstituted and the propylene group is unsubstituted or is substituted with 4-methoxyphenylsulfonylamino, N-phenylureidomethyl, methyl, benzoylaminomethyl, phenyl, 3-phenoxybenzoylaminomethyl, N-phenylureido, phenylsulfonylaminomethyl, 9-fluorenylmethoxy-carbonylaminomethyl, phenoxycarbonylaminomethyl, iso-butoxy-carbonylamino, hydroxycarbonylmethyl, hydroxycarbonylmethoxy, 2-propen-1-yl, N-(4-methoxyphenyl)ureido, 3-phenoxybenzoylamino, 4-methoxyphenylmethyl, 9-fluorenylmethoxycarbonylamino, benzyl, 4-methoxybenzoylamino, benzoylamino, 3,4-methylenedioxybenzoylamino, 4-fluorobenzoylamino, phenylsulfonylamino, 4-phenoxybenzoylamino or amino; or

(ii) R² is selected from CH₂SO₂Me, CH₂SCH₂COOH, CH₂CH₂COOH and CH₂SMe, preferably from CH₂SO₂Me and CH₂SCH₂COOH; and R²² is H;

R³ is i-Pr, cyclohexyl or 1-methyl-1-propyl; and U, U¹, X and Z are as described above.

In other embodiments, the compounds have formula I, where R¹ is C₃₋₁₀ alkyl, or is alkenyl or alkynyl, preferably n-Pr, allyl or propynyl, more preferably n-Pr or propynyl, most preferably n-Pr, and is unsubstituted or substituted with Q;

R² and R²² are selected from (i) or (ii) as follows:

(i) R² and R²² together form propylene or 1,2-dimethylenephenylene, where the 1,2-dimethylenephenylene group is unsubstituted and the propylene group is unsubstituted or is subsituted with 4-methoxyphenylsulfonylamino, N-phenylureidomethyl, methyl, benzoyl-aminomethyl, phenyl, 3-phenoxybenzoylaminomethyl, N-phenylureido, phenylsulfonylaminomethyl, 9-fluorenylmethoxycarbonylaminomethyl, phenoxycarbonylaminomethyl, iso-butoxycarbonylamino, hydroxycarbonylmethyl or hydroxycarbonylmethoxy; or

(ii) R² is selected from CH₂SO₂Me and CH₂SCH₂COOH; and R²² is H;

R³ is i-Pr, cyclohexyl or 1-methyl-1-propyl; and U, U¹, X and Z are as described above.

In more preferred embodiments, R¹ is n-Pr; R² and R²² together form unsubstituted propylene; R³ is i-Pr, cyclohexyl or 1-methyl-1-propyl; and U, U¹, X and Z are selected as described above.

In certain of the preferred embodiments described above, U is —CH— or —C(lower alkyl)— and U¹ is a nitrogen atom. U is more preferably —CH— or —C(Me)—, most preferably —CH—.

Also included in the invention are tautomers, rotamers, enantiomers and other optical isomers of compounds of Formula I, as well as pharmaceutically acceptable salts, solvates and derivatives thereof.

A further feature of the invention is pharmaceutical compositions containing as active ingredient a compound of Formula I (or its salt, solvate or isomers) together with a pharmaceutically acceptable carrier or excipient.

The invention also provides methods for preparing compounds of Formula I, as well as methods for treating diseases such as, for example, HCV and related disorders. The methods for treating comprise administering to a patient suffering from said disease or diseases a therapeutically effective amount of a compound of Formula I, or pharmaceutical compositions comprising a compound of Formula I.

Also disclosed is the use of a compound of Formula I for the manufacture of a medicament for treating HCV and related disorders.

The compounds provided herein include, but are not limited to, those described in the attached Table 1 (along with their activity as ranges of Kᵢ* values in nanomolar, nM) as well as in the Table following the Examples. In Table 1, HCV continuous assay Ki* ranges are: Category a=1-100 nM; Category b=101-999 nM; Category c≧1000 nM.

Depending upon their structure, the compounds of the invention may form pharmaceutically acceptable salts with organic or inorganic acids, or organic or inorganic bases. Examples of suitable acids for such salt formation are hydrochloric, sulfuric, phosphoric, acetic, citric, malonic, salicylic, malic, fumaric, succinic, ascorbic, maleic, methanesulfonic and other mineral and carboxylic acids well known to those skilled in the art. For formation of salts with bases, suitable bases are, for example, NaOH, KOH, NH₄OH, tetraalkylammonium hydroxide, and the like.

In another embodiment, this invention provides pharmaceutical compositions comprising the inventive peptides as an active ingredient. The pharmaceutical compositions generally additionally comprise a pharmaceutically acceptable carrier diluent, excipient or carrier (collectively referred to herein as carrier materials). Because of their HCV inhibitory activity, such pharmaceutical compositions possess utility in treating hepatitis C and related disorders.

In yet another embodiment, the present invention discloses methods for preparing pharmaceutical compositions comprising the inventive compounds as an active ingredient. In the pharmaceutical compositions and methods of the present invention, the active ingredients will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like.

Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. HCV inhibitory activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and pacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as are conventional in the art for this purpose.

Preferably the administration is orally, subcutaneously or intravenously.

Preferably, the pharmaceutical preparation is in a unit dosage form. In such form, the preparation is subdivided into suitably sized unit doses containing appropriate quantities of the active components, e.g., an effective amount to achieve the desired purpose.

The quantity of the inventive active composition in a unit dose of preparation may be generally varied or adjusted from about 1.0 milligram to about 1,000 milligrams, preferably from about 1.0 to about 950 milligrams, more preferably from about 1.0 to about 500 milligrams, and typically from about 1 to about 250 milligrams, according to the particular application. The actual dosage employed may be varied depending upon the patient's age, sex, weight and severity of the condition being treated. Such techniques are well known to those skilled in the art.

Generally, the human oral dosage form containing the active ingredients can be administered 1 or 2 times per day. The amount and frequency of the administration will be regulated according to the judgment of the attending clinician. A generally recommended daily dosage regimen for oral administration may range from about 1.0 milligram to about 1,000 milligrams per day, in single or divided doses.

Some useful terms are described below:

Capsule—refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or by compaction.

Oral gel—refers to the active ingredients dispersed or solubilized in a hydrophillic semi-solid matrix.

Powder for constitution refers to powder blends containing the active ingredients and suitable diluents which can be suspended in water or juices.

Diluent—refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrant—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

Binder—refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′I-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glident—material that prevents caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Bioavailability—refers to the rate and extent to which the active drug ingredient or therapeutic moiety is absorbed into the systemic circulation from an administered dosage form as compared to a standard or control.

Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

Another embodiment of the invention discloses the use of the pharmaceutical compositions disclosed above for treatment of diseases such as, for example, hepatitis C and the like. The method comprises administering a therapeutically effective amount of the inventive pharmaceutical composition to a patient having such a disease or diseases and in need of such a treatment.

In yet another embodiment, the compounds of the invention may be used for the treatment of HCV in humans in monotherapy mode or in a combination therapy mode such as, for example, in combination with antiviral agents such as, for example, ribavirin and/or interferon such as, for example, α-interferon and the like.

As stated earlier, the invention includes tautomers, rotamers, enantiomers and other stereoisomers of the compounds also. Thus, as one skilled in the art appreciates, some of the inventive compounds may exist in suitable isomeric forms. Such variations are contemplated to be within the scope of the invention.

Another embodiment of the invention discloses a method of making the compounds disclosed herein. The compounds may be prepared by several techniques known in the art. Representative illustrative procedures are outlined in the following reaction schemes. It is to be understood that while the following illustrative schemes describe the preparation of a few representative inventive compounds, suitable substitution of any of both the natural and unnatural amino acids will result in the formation of the desired compounds based on such substitution. Such variations are contemplated to be within the scope of the invention.

Abbreviations which may be found in the examples that follow are:

THF: Tetrahydrofuran

DMF: N,N-Dimethylformamide

EtOAc: Ethyl acetate

AcOH: Acetic acid

HOOBt: 3-Hydroxy-1,2,3-benzotriazin-4(3H)-one

EDCl:1-(3-dimethylaminopropyl)-3-ethylcarbodiimide hydrochloride

NMM: N-Methylmorpholine

ADDP: 1,1′-(Azodicarbobyl)dipiperidine

DEAD: Diethylazodicarboxylate

MeOH: Methanol

EtOH: Ethanol

Et₂O: Diethyl ether

PyBrOP: Bromo-tris-pyrrolidinophosphonium hexafluorophosphate

Bn: Benzyl

Boc: tert-Butyloxycarbonyl

Cbz: Benzyloxycarbonyl

Chx: cyclohexyl

Cp: Cylcopentyidienyl

Ts: p-toluenesulfonyl

Me: Methyl

### General Procedure for Preparation of Compounds

Solid-phase synthesis is useful for the production of small amounts of certain compounds of the present invention. As with the conventional solid-phase synthesis of peptides, reactors for the solid-phase synthesis of peptidyl argininals are comprised of a reactor vessel with at least one surface permeable to solvent and dissolved reagents, but not permeable to synthesis resin of the selected mesh size. Such reactors include glass solid phase reaction vessels with a sintered glass frit, polypropylene tubes or columns with frits, or reactor Kans™ made by Irori Inc., San Diego Calif. The type of reactor chosen depends on volume of solid-phase resin needed, and different reactor types might be used at different stages of a synthesis.

Procedure A: Coupling Reaction:

To the resin suspended in DMF (10-15 mL/gram resin) was added Fmoc-amino acid (1 eq), HOBt (1 eq), TBTU (1 eq) and DIEA (1 eq). The mixture was let to react for 4-48 hours. The reactants were drained and the resin was washed successively with dimethylformamide, dichloromethane, methanol, dichloromethane and diethylether (use 10-15 mL solvent/gram resin). The resin was then dried in vacuo.

Procedure B: Fmoc Deprotection:

The Fmoc-protected resin was treated with 20% piperidine in dimethylformamide (10 mL reagent/g resin) for 30 minutes. The reagents were drained and the resin was washed successively with dimethylformamide, dichloromethane, methanol, dichloromethane and diethyl ether (10 mL solvent/gram resin).

Procedure C: Acetylation with Acetic Anhydride:

The resin was suspended in dimethylformamide. The acetylating reagent, prepared by adding 5 mmol (0.47 mL) acetic anhydride and 5 mmol (0.70 mL) triethylamine to 15 mL Dimethylformamide, was added to the resin and the resin was agitated for 30 minutes. The resin was washed successively with dimethylformamide, dichloromethane, methanol, dichloromethane and diethyl ether (10 mL solvent/gram resin).

Procedure D: Semicarbazone Hydrolysis:

The resin was suspended in the cleavage cocktail (10 mL/g resin) consisting of trifluoroacetic acid: pyruvic acid: dichloromethane: water 9:2:2:1 for 2 hours. The reactants were drained and the procedure was repeated three more times. The resin was washed successively with dichloromethane, water and dichloromethane and dried under vacuum.

Procedure E: HF Cleavage:

The fully protected dried peptide-MBHA resin (50 mg) was placed in an HF vessel containing a small stir bar. Anisole (10% of total volume) was added as a scavenger. In the presence of glutamic acid and cysteine amino acids, thioanisole (10%) and 1,2-ethanedithiol (0.2%) were also added. The HF vessel was then hooked up to the HF apparatus (from Immuno Dynamics, Inc.) and the system was flushed with nitrogen for five minutes. It was then cooled down to -70° C. with a dry ice/isopropanol bath. After 20 minutes, HF was distilled to the desired volume (10 mL HF/g resin). The reaction was let to proceed for one and a half hour at 0° C. Work up consisted of removing all the HF using nitrogen. Dichloromethane was then added to the resin and the mixture was stirred for five minutes. This was followed by the addition of 20% acetic acid in water (4 mL). After stirring for 20 minutes, the resin was filtered using a fritted funnel and the dichloromethane was removed under reduced pressure. Hexane was added to the remaining residue and the mixture was agitated, and the layers separated (this was repeated twice to remove scavengers). Meanwhile, the resin was soaked in 1 mL methanol. The aqueous layer (20% HOAc) was added back to the resin and the mixture was agitated for five minutes and then filtered. The methanol was removed under reduced pressure and the aqueous layer was lyophilized. The peptide was then dissolved in 10-25% methanol (containing 0.1% trifluoroacetic acid) and purified by reverse phase HPLC.

### EXAMPLE I

### Synthesis of Ac-EEVVP-nV-(CO)-GMSYS-Am

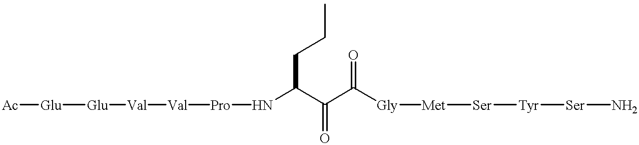

Step I. Synthesis of Fmoc-Met-Ser(tBu)-Tyr(tBu)-Ser(tBu)-MBHA Resin:

MBHA resin (10 g, 4.6 mmol) was placed in a 250 mL fritted reaction vessel equipped with a nitrogen inlet. The resin was neutralized with 5% diisopropylethylamine in dimethylformamide (215 minutes). The resin was then washed twice with 15 mL dimethylformamide followed by three times with 15 mL portions of dichloromethane and dimethylformamide, respectively. a) Fmoc-Ser(tBu)-OH (4.4 g, 11.5 mmol), b) Fmoc-Tyr(tBu)-OH (5.3 g, 11.5 mmol), c) Fmoc-Ser(tBu)-OH (4.4 g, 11.5 mmol) and d) Fmoc-Met-OH (4.3 g, 11.5 mmol) were coupled to the resin, consecutively, following Procedures A and B with 98% overall yield (13.95 g, final resin substitution obtained 0.33 mmol/g).

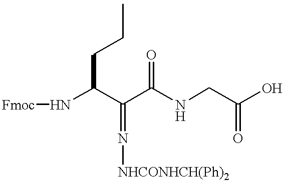

Step II. Synthesis of Fmoc-nVal(dpsc)-Gly-OH (steps a-f below):

a) Synthesis of Allyl Isocyanoacetate

a1) Ethyl isocyanoacetate (96.6 mL, 0.88 mol) was added dropwise to a chilled solution of ethanol (1.5 L) and potassium hydroxide (59.52 g, 1.0 mol). The reaction was slowly warmed to room temperature. After two hours, the precipitated product was filtered on a glass funnel and washed with several portions of chilled ethanol. The potassium salt of isocyanoacetic acid thus obtained was dried in vacuo to a golden-brown solid (99.92 g, 91.8%).

a2) To the product of step a1 (99.92 g, 0.81 mol) dissolved in acetonitrile (810 mL) was added allyl bromide (92 mL, 1.05 mol). After refluxing for four hours, a dark brown solution was obtained. The reaction mixture was concentrated and the remaining residue was picked-up in ether (1.5 L) and washed three times with water (500 ml). The organic layer was dried and concentrated to a dark brown syrup. The crude was purified by vacuum distillation at 7 mm Hg (98vC) to a clear oil (78.92 g, 77.7%). NMR δ ppm (CDCl₃): 5.9 (m, 1 H), 5.3 (m, 2H), 4.7 (d, 2H), 4.25 (s, 2H).

b) Synthesis of 9-fluorenylmethoxycarbonyl-norvalinal (Steps b1-b3 Below)

b1) Synthesis of 9-fluorenylmethoxycarbonyl-norvaline Methyl Ester:

To a chilled solution of Fmoc-norvaline (25 g, 73.75 mmol) in anhydrous methanol (469 mL) was added thionyl chloride (53.76 mL, 0.74 mol) over one hour. Thin layer chromatography in ethylacetate taken an hour later confirmed the completion of the reaction (R_{f}=0.85). The reaction mixture was concentrated and the remaining residue was picked-up in ethylacetate. The organic layer was washed with three 200 ml portions of saturated sodium bicarbonate followed by brine. The organic layer was dried and concentrated to afford the title product as a white solid (26.03 g) in quantitative yield. NMR δ ppm (CD₃OD): 7.7 (m, 2H), 7.6 (m, 2H), 7.4 (m, 2H), 7.3 (m, 2H), 4.3 (m, 2H), 4.1 (m, 2H), 3.7 (s, 3H), 1.7 (m, 1H), 1.6 (m, 1H), 1.4 (m, 2H), 0.95 (t, 3H).

b2) Synthesis of 9-fluorenylmethoxycarbonyl-norvalinol:

To the product of step b1 (26.03 g, 73.75 mmol) in tetrahydrofuran (123 mL) and methanol (246 mL) was added calcium chloride (16.37 g, 147.49 mmol). The reaction mixture was cooled to 0 C and sodium borohydride (11.16 g, 0.3 mol) was added in several batches. Methanol (500 mL) was added to the thick paste obtained and the reaction was stirred at room temperature for 90 minutes. Thin layer chromatography in 2:3 ethylacetate: hexane confirmed the completion of the reaction (R_{f}=0.25). The reaction was quenched with the slow addition of 100 mL water at 0 C. The methanol was removed under reduced pressure and the remaining aqueous phase was diluted with ethylacetate (500 mL). The organic layer was washed three times each with 500 ml portions of water, saturated sodium bicarbonate and brine. The organic layer was dried over sodium sulfate and concentrated to a white solid (21.70 g, 90.5%). NMR δ ppm (CD₃OD): 7.8 (m, 2H), 7.7 (m, 2H), 7.4 (m, 2H), 7.3 (m, 2H), 4.3-4.5 (m, 2H), 4.2 (m, 1H), 3.6 (s, 1H), 3.5 (s, 2H), 1.5 (m, 1H), 1.3-1.4 (m, 3H), 0.99 (m, 3H).

b3) Synthesis of 9-fluorenylmethoxycarbonyl-norvalinal:

To the product of step b2 (21.70 g, 66.77 mmol) in dichloromethane (668 mL) was added triethylamine (37.23 mL, 267.08 mmol) and the solution was cooled to 0 C. A suspension of pyridine sulfur trioxide complex (42.51 g, 267.08 mmol) in dimethylsulfoxide (96 mL) was added to the chilled solution. After one hour, thin layer chromatography in 2:3 ethylacetate: hexane confirmed the completion of the reaction. The dichloromethane was removed under reduced pressure and the remaining residue was picked-up in ethylacetate and washed with several 50 mL portions of water, 1N saturated sodium bisulfate, saturated sodium bicarbonate and brine. The organic layer was concentrated to yield a white solid. Theoretical yield (21.57 g) was assumed and the reaction was taken to the next step without further purification.

c) Synthesis of Fmoc-nVal(CHOH)-Gly-Oallyl

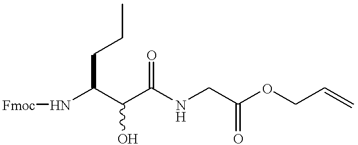

To a solution of Fmoc-norVal-aldehyde obtained from step b3 (5.47 g, 16.90 mmol) in dichloromethane (170 mL) was added allyl isocyanoacetate (step IIa) (2.46 mL, 20.28 mmol) and pyridine (5.47 mL, 67.61 mmol). The reaction mixture was cooled to 0 C and trifluoroacetic acid (3.38 mL, 33.80 mmol) was added dropwise. The reaction was stirred at 0 C for 1 h, and then at room temperature for 48 hours. Thin layer chromatography taken in ethylacetate confirmed the completion of the reaction. The reaction mixture was concentrated and subjected to flash column chromatography using a gradient composed of 20:80 ethylacetate: hexane to 70:30 ethylacetate: hexane. Fractions containing the desired product were pooled and concentrated to a white foam (6.88 g, 87.3%). TLC in 50:50 ethylacetate showed one spot (R_{f}=0.37). NMR δ ppm (CD₃OD): 7.8 (m, 2H), 7.65 (m, 2H), 7.4 (m, 2H), 7.3 (m, 2H), 5.9 (m, 1H), 5.1-5.4 (m, 2H), 4.55-4.65 (m, 2H), 4.3-4.4 (m, 2H), 4.15-4.25 (m, 1H), 4.01 (s, 1H), 3.9-4.0 (m, 3H), 1.5-1.6 (m, 2H), 1.35-1.45 (m, 3H), 0.9 (m, 3H).

d) Synthesis of Fmoc-nVal(CO)-Gly-Oallyl

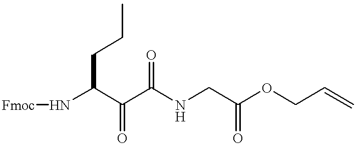

Under a stream of nitrogen, the compound of step c (5.01 g, 10.77 mmol) was dissolved in 100 mL dimethylsulfoxide and 100 mL toluene. Water soluble carbodiimide (EDC, 20.6 g, 107.7 mmol) was then added in one batch. The reaction mixture was cooled to 0 C and dichloroacetic acid (4.44 mL, 53.83 mmol) was added dropwise. After the addition of dichloroacetic acid was completed, the reaction was stirred for 15 minutes at 0 C and 1 h at room temperature. Water (70 mL) was added at 0 C and the toluene was removed under reduced pressure. The remaining residue was diluted with ethylacetate and washed several times with a saturated sodium bicarbonate solution, followed by 1N sodium bisulfate and brine (50 mL portions). The organic layer was dried over sodium sulfate and concentrated. The theoretical yield of 4.99 g was assumed and the reaction was taken to the next step without further purification. Thin layer chromatography in 50:50 ethylacetate: hexane showed one spot (R_{f}=0.73).

e) Synthesis of Fmoc-nVal(dpsc)-Gly-Oallyl (steps e1-e3 below)

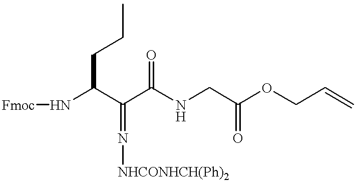

e1) Synthesis of 1-t-Butoxycarbonyl-semicarbazid-4-yl diphenylmethane

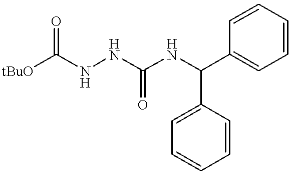

A solution of carbonyldiimidazole (16.2 g, 0.10 mole) in 225 mL of dimethylformamide was prepared at room temperature and allowed to stir under nitrogen. A solution of t-butyl carbazate (13.2 g, 0.100 mol) in 225 mL DMF was then added dropwise over a 30 min. period. Diphenylmethylamine (18.3 g, 0.10 mol) was added next over a 30 min. period. The reaction was allowed to stir at room temperature under nitrogen for one hour. Water (10 mL) was added and the mixture was concentrated to about 150 mL under reduced pressure. This solution was poured into 500 mL water and extracted with 400 mL of ethyl acetate. The ethylacetate phase was extracted two times each with 75 mL 1N HCl, H₂O, saturated sodium bicarbonate solution and sodium chloride, and dried with magnesium sulfate. The mixture was filtered and the solution was concentrated to give 29.5 g (85% yield) of a white foam. This material could be purified by recrystallization from ethyl acetate/hexane, but was pure enough to use directly in the next step: mp 142-143° C. ¹H NMR (CDCl₃) d 1.45 (s, 9H), 6.10 (dd, 2H), 6.42 (s, 1H), 6.67 (bs, 1H), 7.21-7.31 (m, 10H). Anal: Calcd. for C₁₉H₂₃N₃O₃: C, 66.84; H, 6.79; N, 12.31. Found: C, 66.46; H, 6.75; N; 12.90.

e2) Synthesis of diphenylmethyl semicarbazide (dpsc) trifluoroacetate salt

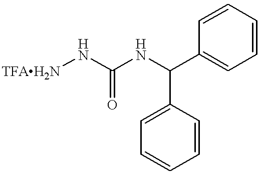

A solution of the product obtained in e1 (3.43 g, 10 mmol) in 12.5 mL of dichloromethane was treated with 12.5 mL of trifluoroacetic acid at room temperature and allowed to stir for 30 min. The solution was added dropwise to 75 mL of ether and the resulting precipitate (2.7 g, 80%) was filtered on a glass funnel. mp 182-184° C. ¹H NMR (CD₃OD) d 6.05 (s, 1H), 7.21-7.35 (m, 10H). ¹³C NMR (CD₃OD) d 57.6, 118.3 (q, CF₃), 126.7, 127.9, 141.6, 156.9, 160.9 (q, CF₃CO₂H).

e3) Synthesis of Fmoc-nVal(dpsc)-Gly-Oallyl

To the product of step IId (4.99 g, 10.75 mmol) dissolved in 130 mL ethanol and 42 mL water were added diphenylmethyl semicarbazide. TFA (obtained in step e2) (7.6 g, 21.5 mmol) and sodium acetate.3H2O (1.76 g, 12.9 mmol), successively. The reaction mixture was refluxed for 90 minutes. The completion of reaction was confirmed by thin layer chromatography taken in 1:1 ethylacetate: hexane. Ethanol was removed under reduced pressure and the remaining residue was picked-up in ethylacetate and washed twice with 10 mL portions of 1N sodium bisulfate, saturated sodium bicarbonate, followed by brine. The organic layer was dried and concentrated and the remaining residue was subjected to flash column chromatography in 20:80 ethylacetate: hexane followed by 50:50 ethylacetate: hexane. Fractions corresponding to the pure product were pulled and concentrated to give a white solid (5.76 g, 78%). Thin layer chromatography in 50:50 ethylacetate: hexane showed two spots (syn and anti isomers) with R_{f}=0.42 and 0.5, respectively.

f) Synthesis of Fmoc-nVal(dpsc)-Gly-OH

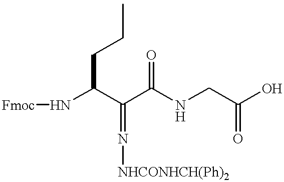

To the product of step II e3 (4.53 g, 6.59 mmol) in THF (300 mL) was added dimedone (4.62 g, 32.97 mmol) followed by tetrakis(triphenylphosphine)palladium(0) catalyst (0.76 g, 0.66 mmol). The completion of the reaction was confirmed after 90 minutes using a 9:1 dichloromethane: methanol thin layer chromatographic system. The reaction mixture was concentrated and the remaining residue was picked-up in ethylacetate and extracted three times with 50 mL portions of 0.1M potassium biphosphate. The organic layer was then treated with 50 mL sodium bisulfite and the two phase system was stirred for 15 minutes. The phases were separated and the procedure was repeated twice more. The organic layer was dried and concentrated and subjected to flash column chromatography starting with 20:80 ethylacetate: hexane and gradually increasing the ethylacetate concentration to 100%. This was followed with 9:1 dichloromethane: methanol solution. The fractions corresponding to the pure product were pooled and concentrated to obtain a white solid (3.99 g, 94%). Thin layer chromatography in 9:1 dichloromethane: methanol showed two spots (syn and anti isomers) with R_{f}=0.03 and 0.13, respectively. NMR δ ppm (CD₃OD): 7.75 (m, 2H), 7.6 (m, 3H), 7.2-7.4 (m, 14H), 6.1-6.2 (m, 1H), 4.25-4.4 (m, 2H), 4.1-4.2 (m, 2H), 3.85 (s, 2H), 1.6-1.8 (m, 2H), 1.3-1.5 (m, 2H), 0.95 (t, 3H).

Step III. Synthesis of Ac-Glu(OtBu)-Glu(OtBu)-Val-Val-Pro-OH:

a) Synthesis of Fmoc-Val-Pro-2ClTrt Resin

In a 1L solid phase reaction vessel equipped with a nitrogen inlet, 25 g of Pro-2ClTrt resin (200-400 mesh, 0.64 mmol/g substitution) was suspended in dimethylformamide (213 mL). Fmoc-Val-OH (1.5 g, 32 mmol) was coupled for four hours according to Procedure A. A small aliquot was taken for calorimetric ninhydrin analysis which showed a 99.5% coupling efficiency in the production of the title compound.

b) Synthesis of Fmoc-Val-Val-Pro-2ClTrt Resin

The resin from the previous step (0.53 mmol/g) was deprotected according to Procedure B. It was then coupled to Fmoc-Val-OH (10.85 g, 32 mmol) according to Procedure A with 99.5% efficiency.

c) Synthesis of Fmoc-Glu(OtBu)-Val-Val-Pro-2ClTrt Resin

The resin from the previous step (0.504 mmol/g) was deprotected according to Procedure B. It was then coupled to Fmoc-Glu(OtBu)-OH (13.63 g, 32 mmol) according to Procedure A with 99.4% efficiency.

d) Synthesis of Fmoc-Glu(OtBu)-Glu(OtBu)-Val-Val-Pro-2ClTrt Resin

The resin from the previous step (0.461 mmol/g) was deprotected according to Procedure B. It was then coupled to Fmoc-Glu(OtBu)-OH (13.63 g, 32 mmol) according to procedure A with 99.2% efficiency to yield the titled compound.

e) Synthesis of Ac-Glu(OtBu)-Glu(OtBu)-Val-Val-Pro-2ClTrt Resin

The resin from the previous step (0.42 mmol/g) was deprotected according to procedure B. The N-terminus was then capped according to Procedure C to yield the desired compound in 99.7% efficiency.

f) Synthesis of Ac-Glu(OtBu)-Glu(OtBu)-Val-Val-Pro-OH

The resin from the previous step was transferred to a 1L plastic bottle and cleaved in the presence of 525 ml solution of acetic acid: trifluoroethanol: dichloromethane (1:1:3) with vigorous shaking for two hours. The resin was filtered using a fritted funnel and washed 3×50 mL with dichloromethane. The brownish red filtrate was concentrated to an oil which was then treated three times with 50 ml of a 1:1 mixture of dichloromethane: n-heptane. The crude off-white powder (13 g) was then dissolved in minimum amount of methanol and purified by HPLC using a 2.2×25 cm reverse phase column, containing a C-18 resin comprised of 10 micron size gel particles with a 300 angstrom pore size, eluting with a gradient ranging from 15-55% acetonitrile in water. The pure fractions were pulled and concentrated to a fluffy, white product (7.5 g, 65%). Analytical HPLC using a 4.6×250 mm reverse phase column, containing a C-18 resin comprised of 5 micron size gel particles with a 300 angstrom pore size ran at 5-50% acetonitrile (containing 0.1% trifluoroacetic acid) showed one peak with the retention time of 20.5 min. Low resolution mass spectrum confirmed the desired mass (MH⁺726.5).

Step IV. Synthesis of Fmoc-nVal(dpsc)-Gly-Met-Ser(tBu)-Tyr(tBu)-Ser(tBu)-MBHA:

The resin obtained from step I (2 g, 0.66 mmol) was deprotected according to Procedure B. Fmoc-nVal(dpsc)-Gly-OH (step IIf) (1.1 g, 1.7 mmol) was then coupled over 18 hours according to procedure A using N-methylpyrrolidine as solvent with 98% efficiency (2 g resin obtained, new resin substitution determined to be 0.276 mmol/g).

Step V. Synthesis of Ac-Glu(OtBu)-Glu(OtBu)-Val-Val-Pro-nVal(dpsc)-Gly-Met-Ser(tBu)-Tyr(tBu)-Ser(tBu)-MBHA:

1 g resin (0.28 mmol) from step IV was placed in a fritted reaction vessel. The resin was deprotected according to Procedure B. Ac-Glu(OtBu)-Glu(OtBu)-Val-Val-Pro-OH (400 mg, 0.55 mmol) (obtained in IIIf) was then coupled over 18 hours according to Procedure A with 98% efficiency (978 mg resin obtained).

Step VI. Synthesis of Ac-Glu-Glu-Val-Val-Pro-nVal(CO)-Gly-Met-Ser-Tyr-Ser-MBHA:

The resin from step V (998 mg) was treated for one hour with 10 ml dichloromethane: trifluoroacetic acid (1:1). The reactants were drained and the resin was thoroughly washed with dichloromethane. The resin was subjected to semicarbazone deprotection Procedure D and dried under vacuum to yield 943 mg resin.

Step VII. Synthesis of Ac-Glu-Glu-Val-Val-Pro-nVal(CO)-Gly-Met-Ser-Tyr-Ser-NH2:

The resin obtained from step VI (942.8 mg) was cleaved with HF according to Procedure E. The crude product (314 mg) was subjected to HPLC purification using a 2.2×25 cm reverse phase column, containing a C-18 resin comprised of 10 micron size gel particles with a 300 angstrom pore size, eluting with a gradient using 0-30% (30 minutes) acetonitrile in water followed by 30-75% (10 minutes) acetonitrile in water. The desired fractions were pulled and concentrated to a white solid (238 mg, 26%). Analytical HPLC using a 4.6×250 mm reverse phase column, containing a C-18 resin comprised of 5 micron size gel particles with a 300 angstrom pore size, eluting at 5-50% acetonitrile (containing 0.1% trifluoroacetic acid) showed one peak at 13 minutes. Low resolution mass spectrum confirmed the desired mass (MH⁺1265.6). The Table below lists the synthesis of other similar compounds:

Table of 11mer Compounds Synthesized according to Example 1

| COMPOUND NAME | SYNTHESIS |
|---|---|
| AcEEVVPnV-(CO)-GMSYS-Am | example I |
| AcEEVVPnV-CO-GMdSYS-Am | step Ic: used Fmoc-dSer(tBu)-OH |
| AcEEVVPnV-CO-GMdHYS-Am | step Ic: used Fmoc-dHis(Trt)-OH |
| AcEEVVPnV-CO-GMdDYS-Am | step Ic: used Fmoc-dAsp(tBu)-OH |
| AcEEVVPnV-CO-GdMSYS-Am | step Id: used Fmoc-dMet-OH |
| AcEEVVPnV-CO-GdMdSYS-Am | step Ic: used Fmoc-Ser(tBu)-OH, |
| | step Id: used Fmoc-dMet-OH |
| AcEEVVPnV-CO-GdMHYS-Am | step Ic: used Fmoc-His(Trt)-OH, |
| | step Id: used Fmoc-dMet-OH |
| AcEEVVPnV-CO-GdMDYS-Am | step Ic: used Fmoc-Asp(OtBu)-OH, |
| | step Id: used Fmoc-dMet-OH |
| AcEEVVPnV-CO-GdMdDYS-Am | step Ic: used Fmoc-dAsp(OtBu)-OH, |
| | step Id: used Fmoc-dMet-OH |
| AcEEVVPnV-CO-GGSYS-Am | step Id: used Fmoc-Gly-OH |
| AcEEVVPnV-CO-GGHYS-Am | step Ic: used Fmoc-His(Trt)-OH, |
| | step Id: used Fmoc-Gly-OH |
| AcEEVVPnV-CO-GGdHYS-Am | step Ic: used Fmoc-dHis(Trt)-OH, |
| | step Id: used Fmoc-Gly-OH |
| AcEEVVPnV-CO-GGDYS-Am | step Ic: used Fmoc-Asp(OtBu)-OH, |
| | step Id: used Fmoc-Gly-OH |
| AcEEVVPnV-CO-GGdDYS-Am | step Ic: used Fmoc-dAsp(OtBu)-OH, |
| | step Id: used Fmoc-Gly-OH |
| AcEEVVPnV-CO-GQSYS-Am | step Id: used Fmoc-Gln(Trt)-OH |
| AcEEVVPnV-CO-GQdSYS-Am | step Ic: used Fmoc-dSer(tBu)-OH, |
| | step Id: used Fmoc-Gln(Trt)-OH |
| AcEEVVPnV-CO-GQdHYS-Am | step Ic: used Fmoc-dHis(Trt)-OH, |
| | step Id: used Fmoc-Gln(Trt)-OH |
| AcEEVVPnV-CO-GQdDYS-Am | step Ic: used Fmoc-dAsp(OtBu)-OH, |
| | step Id: used Fmoc-Gln(Trt)-OH |
| AcEEVVPnV-CO-GdQSYS-Am | step Id: used Fmoc-dGln(Trt)-OH |
| AcEEVVPnV-CO-GdQdSYS-Am | step Ic: used Fmoc-dSer(tBu)-OH, |
| | step Id: used Fmoc-dGln(Trt)-OH |
| AcEEVVPnV-CO-GdQHYS-Am | step Ic: used Fmoc-His(Trt)-OH, |
| | step Id: used Fmoc-dGln(Trt)-OH |
| AcEEVVPnV-CO-GdQDYS-Am | step Ic: used Fmoc-Asp(OtBu)-OH, |
| | step Id: used Fmoc-dGln(Trt)-OH |
| AcEEVVPnV-CO-GdQdDYS-Am | step Ic: used Fmoc-dAsp(OtBu)-OH, |
| | step Id: used Fmoc-dGln(Trt)-OH |
| AcEEVVPnV-CO-GTSYS-Am | step Id: used Fmoc-Thr(tBu)-OH |
| AcEEVVPnV-CO-GTdSYS-Am | step Ic: used Fmoc-dSer(tBu)-OH, |
| | step Id: used Fmoc-Thr(tBu)-OH |
| AcEEVVPnV-CO-GTHYS-Am | step Ic: used Fmoc-His(Trt)-OH, |
| | step Id: used Fmoc-Thr-OH |
| AcEEVVPnV-CO-GTDYS-Am | step Ic: used Fmoc-Asp(OtBu)OH, |
| | step Id: use Fmoc-Thr(tBu)-OH |
| AcEEVVPnV-CO-GTdDYS-Am | step Ic: used Fmoc-dAsp(OtBu)-OH, |
| | step Id: used Fmoc-Thr(tBu)-OH |
| AcEEVVPnV-CO-GSdSYS-Am | step Ic: used Fmoc-dSer(tBu)-OH, |
| | step Id: used Fmoc-Ser(tBu)-OH |
| AcEEVVPnV-CO-GSdHYS-Am | step Ic: used Fmoc-dHis(Trt)-OH, |
| | step Id: used Fmoc-Ser(tBu)-OH |
| AcEEVVPnV-CO-GSdDYS-Am | step Ic: used Fmoc-dAsp(OtBu)-OH, |
| | step Id: used Fmoc-Ser(tBu)-OH |
| AcEEVVPnV-CO-GdSSYS-Am | step Id: used Fmoc-dSer(tBu)-OH |
| AcEEVVPnV-CO-GdSdSYS-Am | step Ic: used Fmoc-dSer(tBu)-OH, |
| | step Id: used Fmoc-d-Ser(tBu)-OH |
| AcEEVVPnV-CO-GdSHYS-Am | step Ic: used Fmoc-His(Trt)-OH, |
| | step Id: used Fmoc-dSer(tBu)-OH |
| AcEEVVPnV-CO-GdSdHYS-Am | step Ic: used Fmoc-dHis(Trt)-OH, |
| | step Id: used Fmoc-dSer(tBu)-OH |
| AcEEVVPnV-CO-GdSDYS-Am | step Ic: used Fmoc-Asp(OtBu)-OH, |
| | step Id: used Fmoc-dSer(tBu)-OH |
| AcEEVVPnV-CO-GdSdDYS-Am | step Ic: used Fmoc-dAsp(OtBu)-OH, |
| | step Id: used Fmoc-dSer(tBu)-OH |
| AcEEVVPnV-CO-GM(O)HYS-Am | step IC: used Fmoc-His(Trt)-OH |
| AcEEVVPnV-(CO)-GdM(O)SYS-Am | step Id: used Fmoc-dMet-OH |
| AcEEVVPnV-CO-GdM(O)dHYS-Am | step Ic: used Fmoc-dHis(Trt)-OH, |
| | step Id: used Fmoc-dMet-OH |
| AcEEVVPnV-CO-GdM(O)DYS-Am | step Ic: used Fmoc-Asp(OtBu)-OH, |
| | step Id: used Fmoc-dMet-OH |
| AcEEVVPnV-CO-GdM(O)dDYS-Am | step Ic: used Fmoc-dAsp(OtBu)-OH, |
| | step Id: used Fmoc-dMet-OH |
| Ac-EEVVP-V-(CO)-GMSYS-Am | step II (b1): used Fmoc-Val-OH |
| Ac-EEVVP-L-(CO)-GMSYS-Am | step II (b1): used Fmoc-Leu-OH |
| Ac-EEVVP-nL-(CO)-GMSYS-Am | step II (b1): used Fmoc-nLeu-OH |
| Ac-EEVVP-Abu-(CO)-GMSYS-Am | step II (b1): used Fmoc-Abu-OH |
| Ac-EEVVP-(s,s)alloT-(CO)-GMSYS-Am | step II (b1): used Fmoc-(s,s)alloThr- |
| | OH |
| Ac-EEVVP-G(propynyl)-(CO)-GMSYS- | step II b1 used Fmoc-G(propynyl)-OH |
| Am | |

Assay for HCV Protease Inhibitory Activity:

Spectrophotometric Assay:

Spectrophotometric assay for the HCV serine protease was performed on the inventive compounds by following the procedure described by R. Zhang et al, Analytical Biochemistry, 270 (1999) 268-275, the disclosure of which is incorporated herein by reference. The assay based on the proteolysis of chromogenic ester substrates is suitable for the continuous monitoring of HCV NS3 protease activity. The substrates were derived from the P side of the NS5A-NS5B junction sequence (Ac-DTEDVVX(Nva), where X=A or P) whose C-terminal carboxyl groups were esterified with one of four different chromophoric alcohols (3- or 4-nitrophenol, 7-hydroxy-4-methyl-coumarin, or 4-phenylazophenol). Presented below are the synthesis, characterization and application of these novel spectrophotometric ester substrates to high throughput screening and detailed kinetic evaluation of HCV NS3 protease inhibitors.

Materials and Methods:

Materials:

Chemical reagents for assay related buffers were obtained from Sigma Chemical Company (St. Louis, Mo.). Reagents for peptide synthesis were from Aldrich Chemicals, Novabiochem (San Diego, Calif.), Applied Biosystems (Foster City, Calif.) and Perseptive Biosystems (Framingham, Mass.). Peptides were synthesized manually or on an automated ABI model 431A synthesizer (from Applied Biosystems). UVNIS Spectrometer model LAMBDA 12 was from Perkin Elmer (Norwalk, Conn.) and 96-well UV plates were obtained from Corning (Corning, N.Y.). The prewarming block was from USA Scientific (Ocala, Fla.) and the 96-well plate vortexer was from Labline Instruments (Melrose Park, Ill.). A Spectramax Plus microtiter plate reader with monochrometer was obtained from Molecular Devices (Sunnyvale, Calif.).

Enzyme Preparation:

Recombinant heterodimeric HCV NS3/NS4A protease (strain 1a) was prepared by using the procedures published previously (D. L. Sali et al, Biochemistry, 37 (1998) 3392-3401). Protein concentrations were determined by the Biorad dye method using recombinant HCV protease standards previously quantified by amino acid analysis. Prior to assay initiation, the enzyme storage buffer (50 mM sodium phosphate pH 8.0, 300 mM NaCl, 10% glycerol, 0.05% lauryl maltoside and 10 mM DTT) was exchanged for the assay buffer (25 mM MOPS pH 6.5, 300 mM NaCl, 10% glycerol, 0.05% lauryl maltoside, 5 μM EDTA and 5 μM DTT) utilizing a Biorad Bio-Spin P-6 prepacked column.

Substrate Synthesis and Purification:

The synthesis of the substrates was done as reported by R. Zhang et al, (ibid.) and was initiated by anchoring Fmoc-Nva-OH to 2-chlorotrityl chloride resin using a standard protocol (K. Barlos et al, Int. J. Pept. Protein Res., 37 (1991), 513-520). The peptides were subsequently assembled, using Fmoc chemistry, either manually or on an automatic ABI model 431 peptide synthesizer. The N-acetylated and fully protected peptide fragments were cleaved from the resin either by 10% acetic acid (HOAc) and 10% trifluoroethanol (TFE) in dichloromethane (DCM) for 30 min, or by 2% trifluoroacetic acid (TFA) in DCM for 10 min. The combined filtrate and DCM wash was evaporated azeotropically (or repeatedly extracted by aqueous Na₂CO₃ solution) to remove the acid used in cleavage. The DCM phase was dried over Na₂SO₄ and evaporated.

The ester substrates were assembled using standard acid-alcohol coupling procedures (K. Holmber et al, Acta Chem. Scand., B33 (1979) 410-412). Peptide fragments were dissolved in anhydrous pyridine (30-60 mg/ml) to which 10 molar equivalents of chromophore and a catalytic amount (0.1 eq.) of para-toluenesulfonic acid (pTSA) were added. Dicyclohexylcarbodiimide (DCC, 3 eq.) was added to initiate the coupling reactions. Product formation was monitored by HPLC and found to be complete following 12-72 hour reaction at room temperature. Pyridine solvent was evaporated under vacuum and further removed by azeotropic evaporation with toluene. The peptide ester was deprotected with 95% TFA in DCM for two hours and extracted three times with anhydrous ethyl ether to remove excess chromophore. The deprotected substrate was purified by reversed phase HPLC on a C3 or C8 column with a 30% to 60% acetonitrile gradient (using six column volumes). The overall yield following HPLC purification was approximately 20-30%. The molecular mass was confirmed by electrospray ionization mass spectroscopy. The substrates were stored in dry powder form under desiccation.

Spectra of Substrates and Products:

Spectra of substrates and the corresponding chromophore products were obtained in the pH 6.5 assay buffer. Extinction coefficients were determined at the optimal off-peak wavelength in 1-cm cuvettes (340 nm for 3-Np and HMC, 370 nm for PAP and 400 nm for 4-Np) using multiple dilutions. The optimal off-peak wavelength was defined as that wavelength yielding the maximum fractional difference in absorbance between substrate and product (product OD−substrate OD)/substrate OD).

Protease Assay:

HCV protease assays were performed at 30° C. using a 200 μl reaction mix in a 96-well microtiter plate. Assay buffer conditions (25 mM MOPS pH 6.5, 300 mM NaCl, 10% glycerol, 0.05% lauryl maltoside, 5 μM EDTA and 5 μM DTT) were optimized for the NS3/NS4A heterodimer (D. L. Sali et al, ibid.)). Typically, 150 μl mixtures of buffer, substrate and inhibitor were placed in wells (final concentration of DMSO 4% v/v) and allowed to preincubate at 30° C. for approximately 3 minutes. Fifty μls of prewarmed protease (12 nM, 30° C.) in assay buffer, was then used to initiate the reaction (final volume 200 μl). The plates were monitored over the length of the assay (60 minutes) for change in absorbance at the appropriate wavelength (340 nm for 3-Np and HMC, 370 nm for PAP, and 400 nm for 4-Np) using a Spectromax Plus microtiter plate reader equipped with a monochrometer (acceptable results can be obtained with plate readers that utilize cutoff filters). Proteolytic cleavage of the ester linkage between the Nva and the chromophore was monitored at the appropriate wavelength against a no enzyme blank as a control for non-enzymatic hydrolysis. The evaluation of substrate kinetic parameters was performed over a 30-fold substrate concentration range (˜6-200 μM). Initial velocities were determined using linear regression and kinetic constants were obtained by fitting the data to the Michaelis-Menten equation using non-linear regression analysis (Mac Curve Fit 1.1, K. Raner). Turnover numbers (k_{cat}) were calculated assuming the enzyme was fully active.

Evaluation of Inhibitors and Inactivators:

The inhibition constants (Kᵢ) for the competitive inhibitors Ac-D-(D-Gla)-L-I-(Cha)-C—OH (27), Ac-DTEDVVA(Nva)-OH and Ac-DTEDVVP(Nva)-OH were determined experimentally at fixed concentrations of enzyme and substrate by plotting vₒ/vᵢ vs. inhibitor concentration ([I]ₒ) according to the rearranged Michaelis-Menten equation for competitive inhibition kinetics: vₒ/vᵢ=1+[I]ₒ/(Kᵢ(1+[S]ₒ/Kₘ)), where vₒ is the uninhibited initial velocity, vᵢ is the initial velocity in the presence of inhibitor at any given inhibitor concentration ([I]ₒ) and [S]ₒ is the substrate concentration used. The resulting data were fitted using linear regression and the resulting slope, 1/(Kᵢ(1+[S]ₒ/Kₘ), was used to calculate the Kᵢ* value.

The obtained Kᵢ* values for the various compounds of the present invention are given in the afore-mentioned Table wherein the compounds have been arranged in the order of ranges of Kᵢ* values. From these test results, it would be apparent to the skilled artisan that the compounds of the invention have excellent utility as NS3-serine protease inhibitors.

While the present invention has been described with in conjunction with the specific embodiments set forth above, many alternatives, modifications and other variations thereof will be apparent to those of ordinary skill in the art. All such alternatives, modifications and variations are intended to fall within the spirit and scope of the present invention.

TABLE 1

| | | HCV Ki* |
|---|---|---|
| STRUCTURE | NAME | (nM) |
| 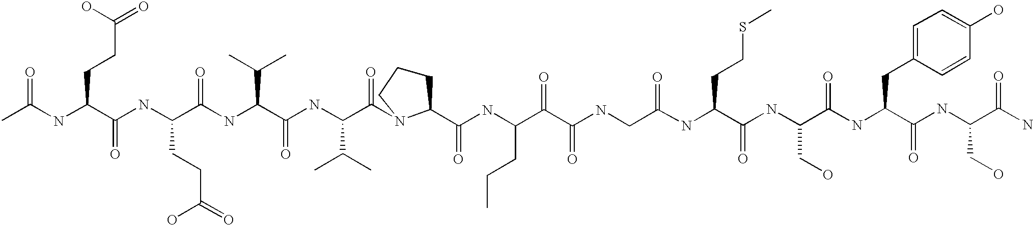 | Ac-EEVVP-nV-(CO)-GMSY | 2.8 |
| 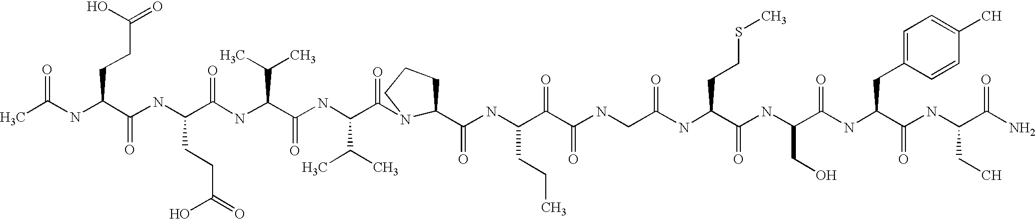 | AcEEVVP-nV-(CO)- GMdSYS-Am | 38 |
| 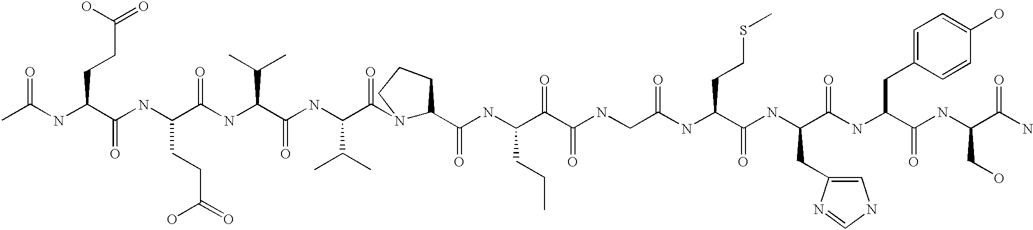 | AcEEVVP-nV-(CO)- GMdHYS-Am | 86 |
| 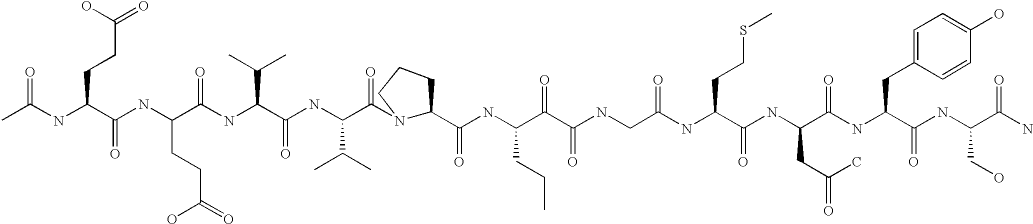 | AcEEVVP-nV-(CO)- GMdDYS-Am | 38 |
| 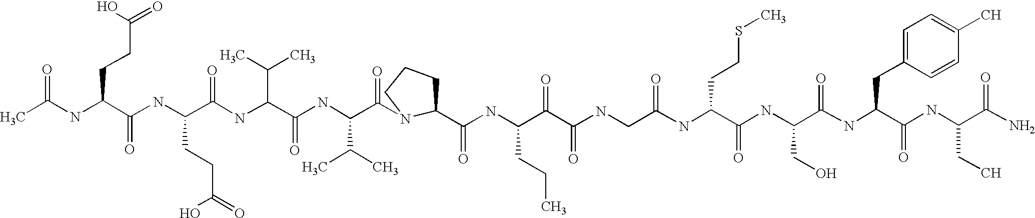 | AcEEVVP-nV-(CO)- GdMSYS-Am | 120 |
| 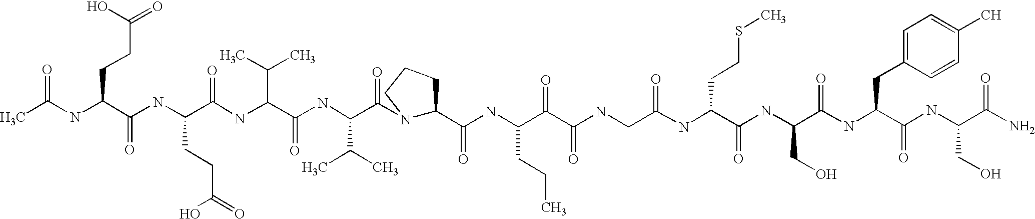 | AcEEVVP-nV-(CO)- GdMdSYS-Am | 120 |
| 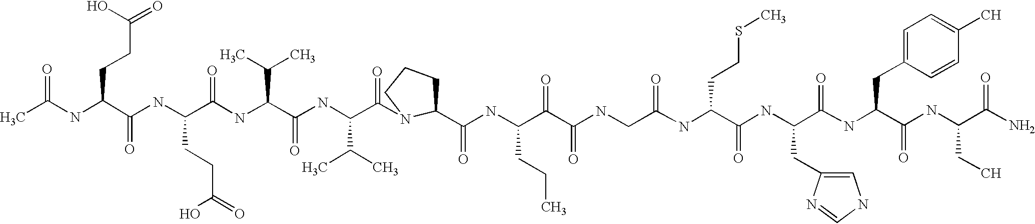 | AcEEVVP-nV-(CO)- GdMHYS-Am | 120 |
| 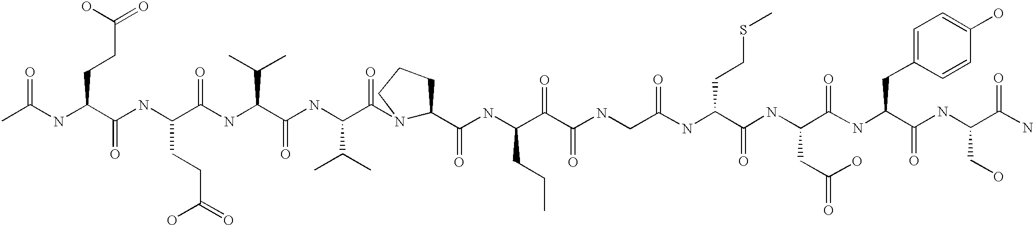 | AcEEVVP-nV-(CO)- GdMDYS-Am | 61 |
| 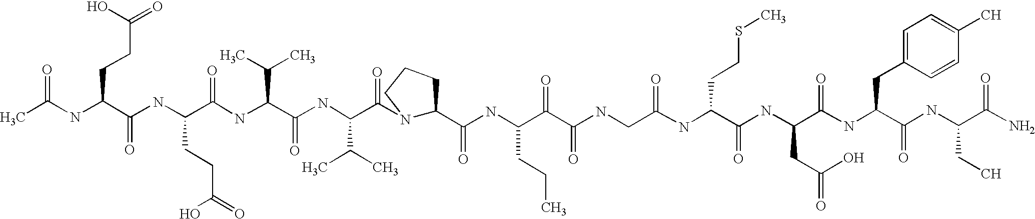 | AcEEVVP-nV-(CO)- GdMdDYS-Am | 87 |
| 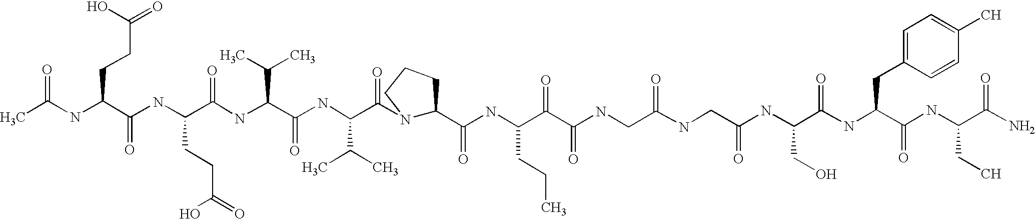 | AcEEVVP-nV-(CO)- GGSYS-Am | 20 |
| 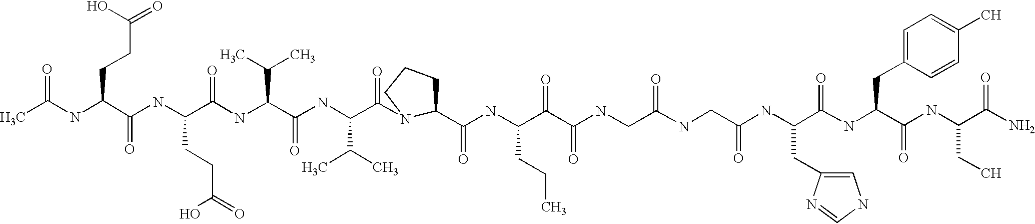 | AcEEVVP-nV-(CO)- GGHYS-Am | 50 |
| 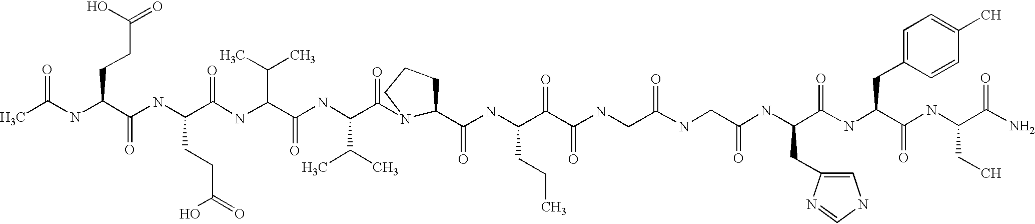 | AcEEVVP-Nv-(CO)- GGdHYS-Am | 120 |
| 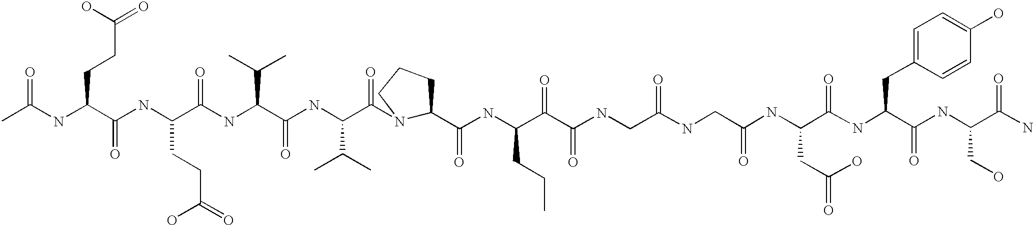 | AcEEVVP-nV-(CO)- GGDYS-Am | 9.6 |
| 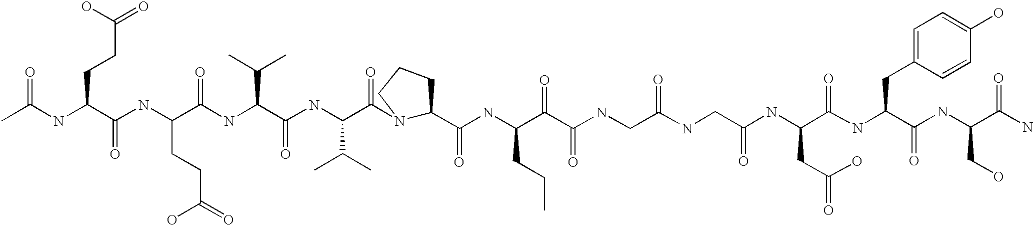 | AcEEVVP-nV-(CO)- GGdDYS-Am | 39 |
| 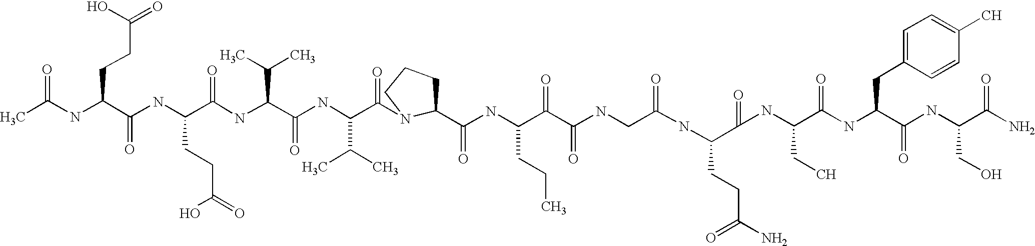 | AcEEVVP-nV-(CO)- GQSYS-Am | 4.3 |
| 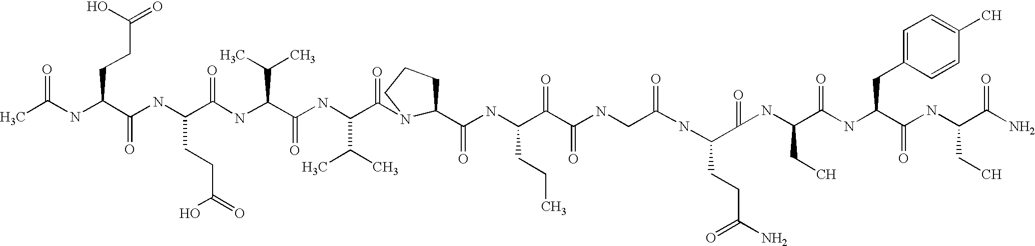 | AcEEVVP-nV-(CO)- GQdSYS-Am | 29 |
| 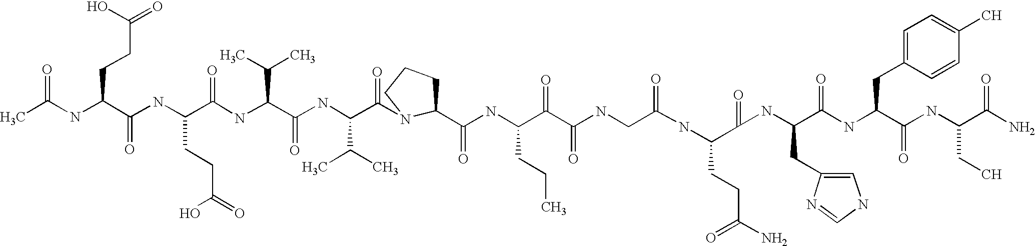 | AcEEVVP-nV-(CO)- GQdHYS-Am | 50 |
| 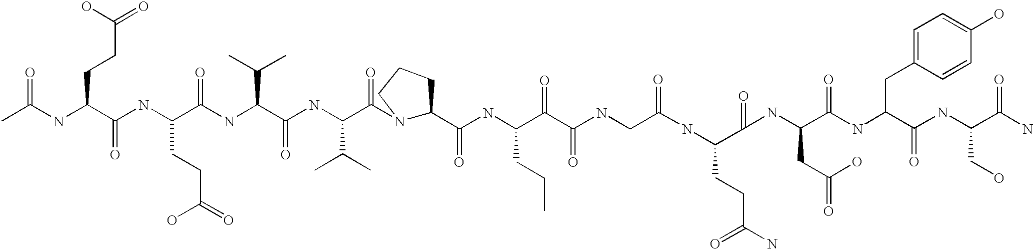 | AcEEVVP-nV-(CO)- GQdDYS-Am | 40 |
| 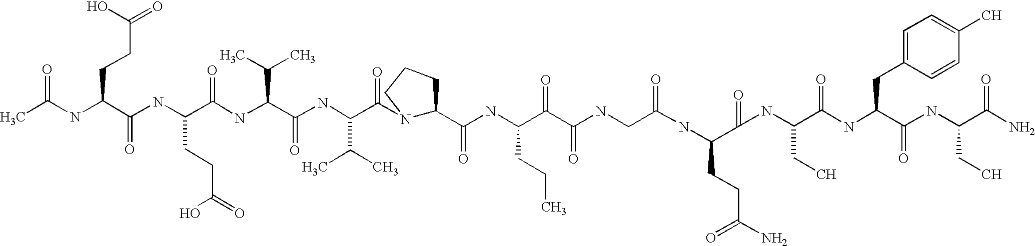 | AcEEVVP-nV-(CO)- GdQSYS-Am | 64 |
| 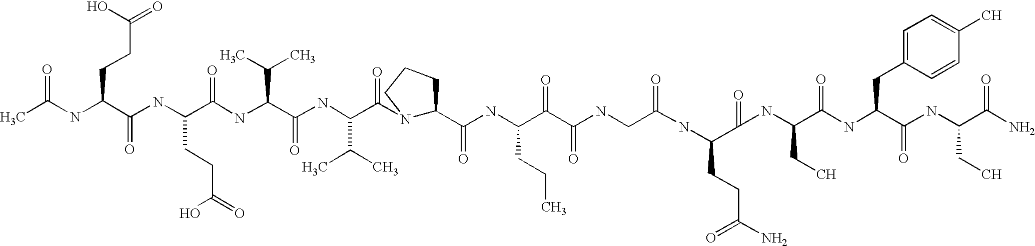 | AcEEVVP-nV-(CO)- GdQdSYS-Am | 87 |
| 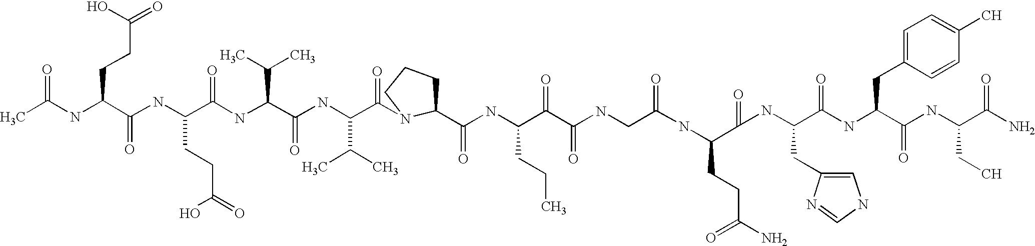 | AcEEVVP-nV-(CO)- GdQHYS-Am | 63 |
| 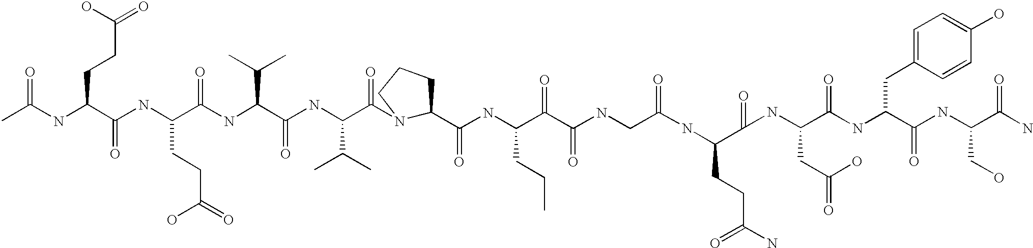 | AcEEVVP-nV-(CO)- GdQDYS-Am | 44 |
| 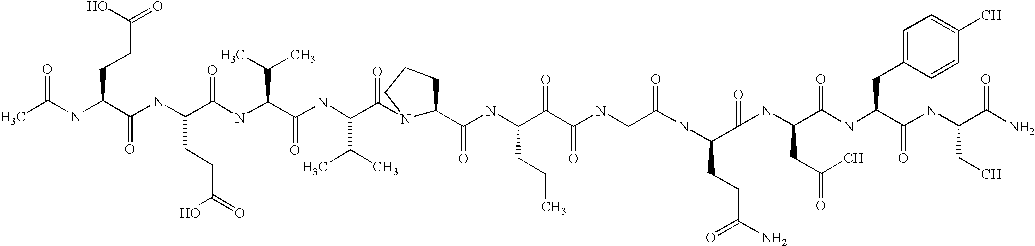 | AcEEVVP-nV-(CO)- GdQdDYS-Am | 39 |
| 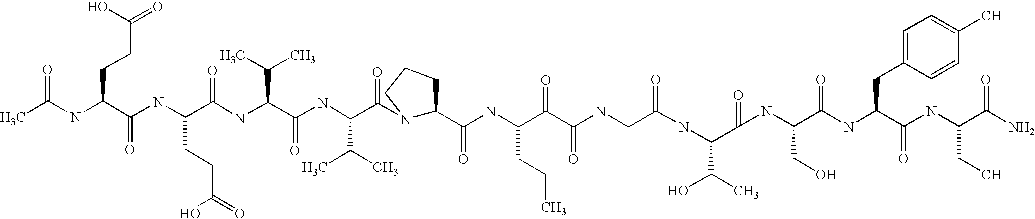 | AcEEVVP-nV-(CO)- GTSYS-Am | 2.2 |
| 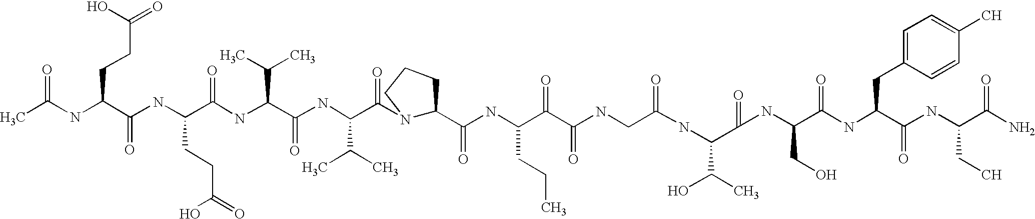 | AcEEVVP-nV-(CO)- GTdSYS-Am | 10.8 |
| 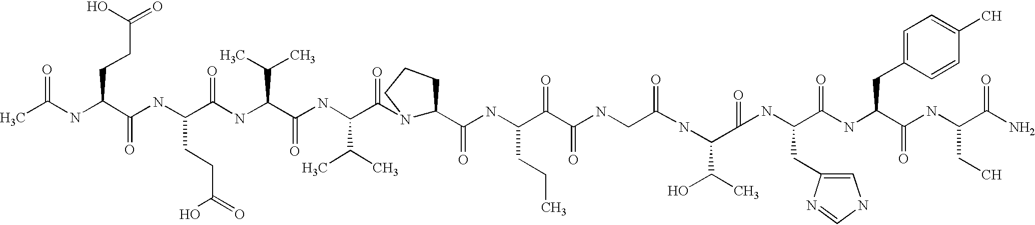 | AcEEVVP-nV-(CO)- GTHYS-Am | 3 |
| 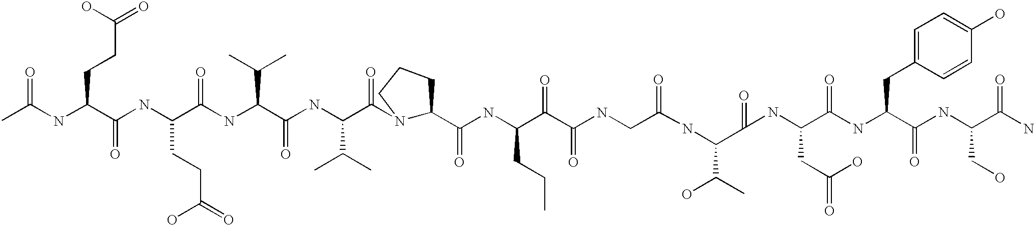 | AcEEVVP-nV-(CO)- GTDYS-Am | 2.7 |
| 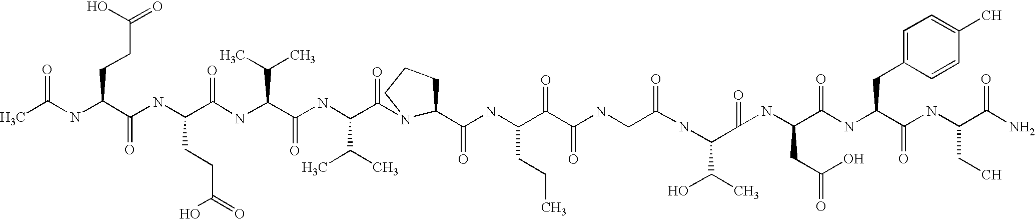 | AcEEVVP-nV-(CO)- GTdDYS-Am | 9.1 |
| 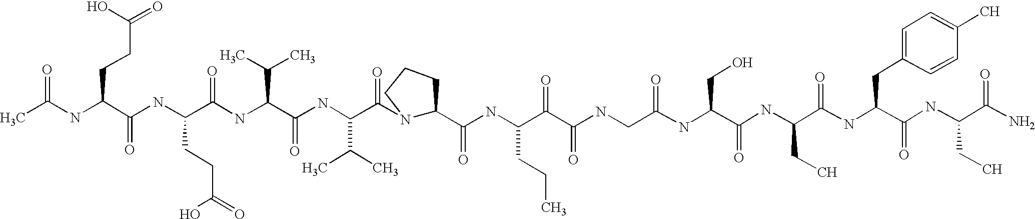 | AcEEVVP-nV-(CO)- GSdSYS-Am | 27.2 |
| 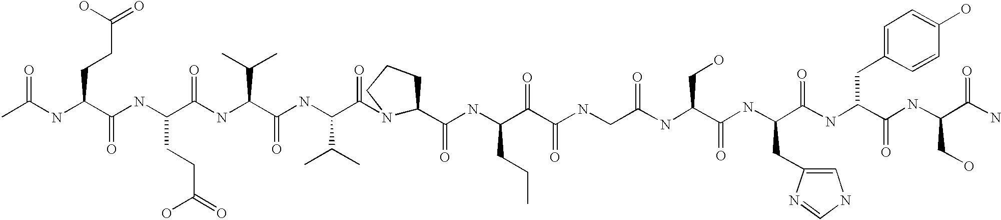 | AcEEVVP-nV-(CO)- GSdHYS-Am | 29.1 |
| 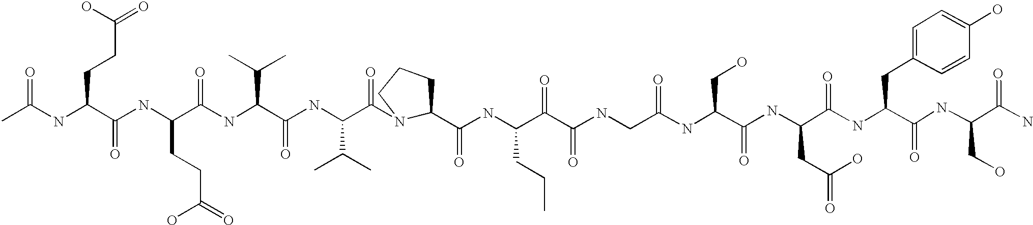 | AcEEVVP-nV-(CO)- GSdDYS-Am | 24.2 |
| 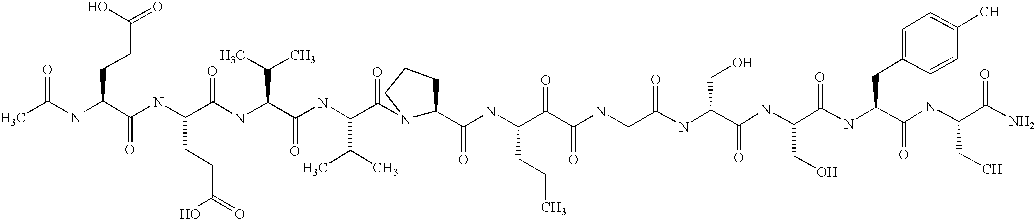 | AcEEVVP-nV-(CO)- GdSSYS-Am | 39.4 |
| 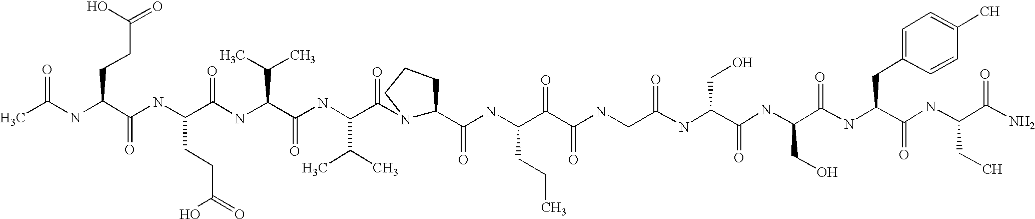 | AcEEVVP-nV-(CO)- GdSdSYS-Am | 32.3 |
| 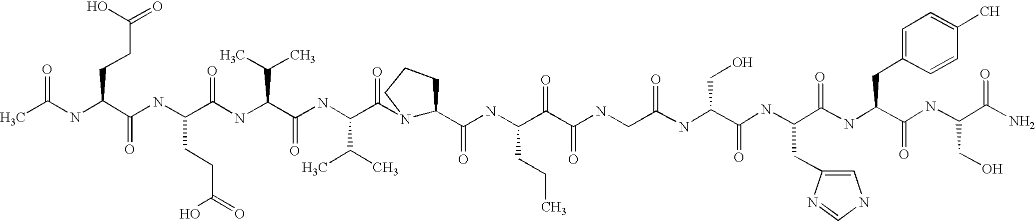 | AcEEVVP-nV-(CO)- GdSHYS-Am | 30 |
| 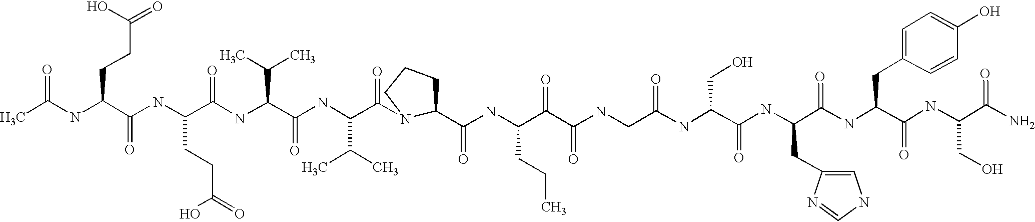 | AcEEVVP-nV-(CO)- GdSdHYS-Am | 57.6 |
| 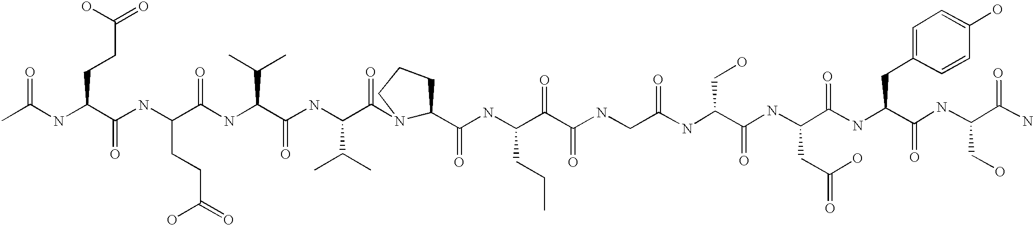 | AcEEVVP-nV-(CO)- GdSDYS-Am | 17 |
| 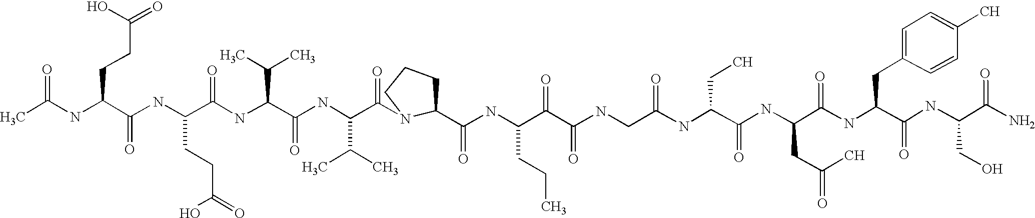 | AcEEVVP-nV-(CO)- GdSdDYS-Am | 11 |
| 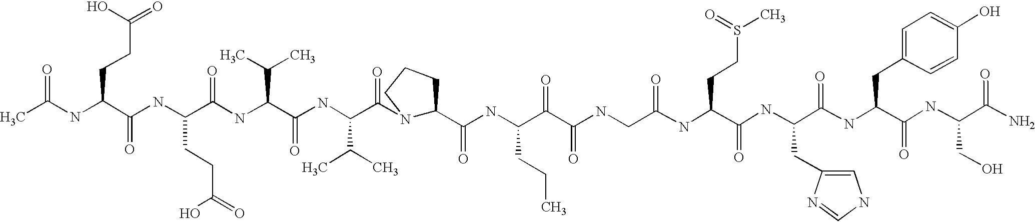 | AcEEVVP-nV-(CO)- GM(O)HYS-Am | 4.1 |
| 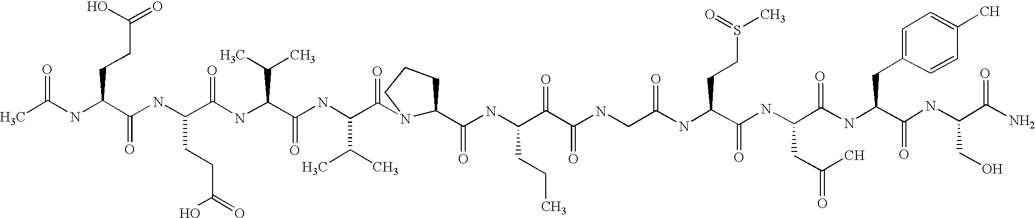 | AcEEVVP-nV-(CO)- GdM(O)SYS-Am | 3.5 |
| 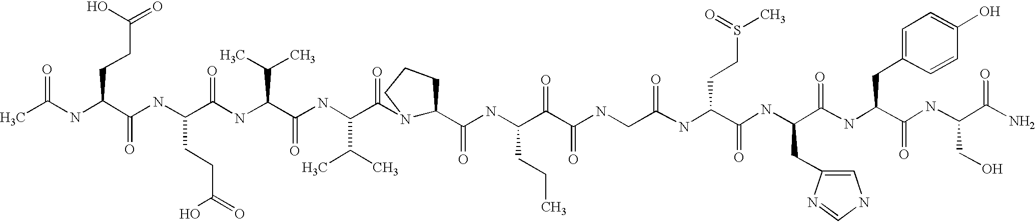 | AcEEVVP-nV-(CO)- GdM(O)dHYS-Am | 330 |
| 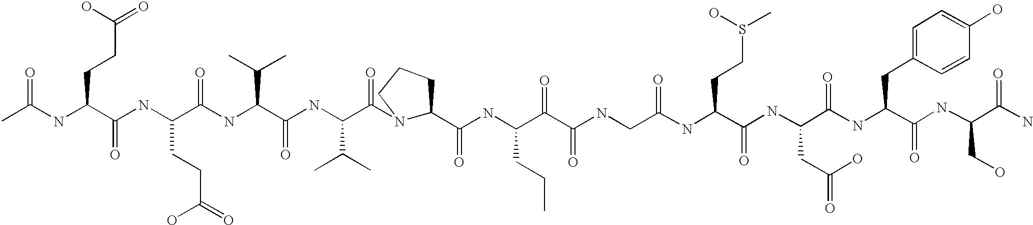 | AcEEVVP-nV-(CO)- GdM(O)DYS-Am | 83 |
| 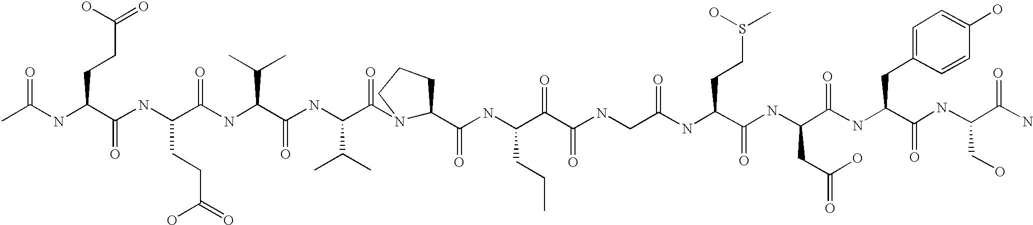 | AcEEVVP-nV-(CO)- GdM(O)dDYS-Am | 60 |
| 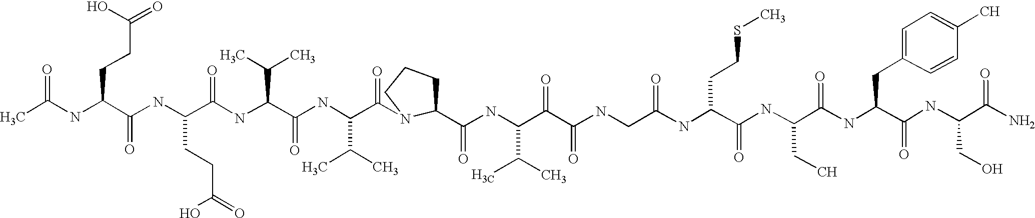 | Ac-EEVVP-V-(CO)- GMSYS-Am | 130 |
| 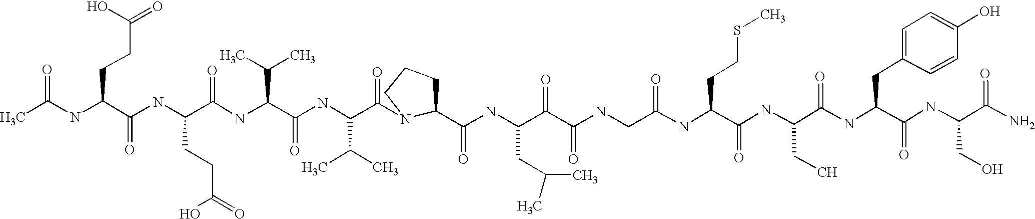 | Ac-EEVVP-L-(CO)- GMSYS-Am | 66 |
| 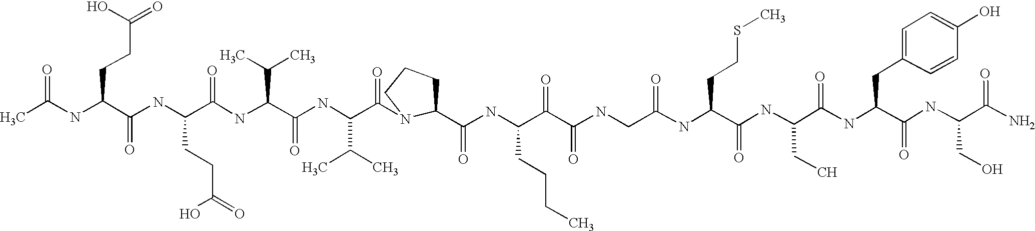 | Ac-EEVVP-nL-(CO)- GMSYS-Am | 110 |
| 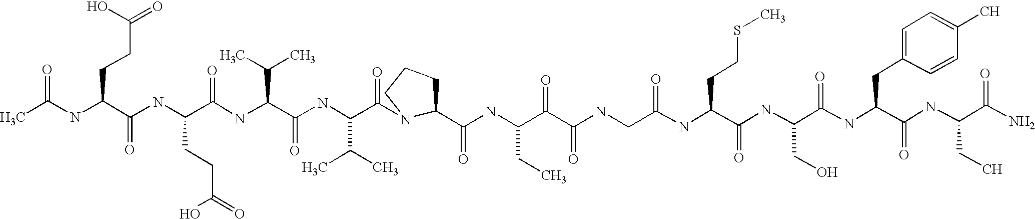 | Ac-EEVVP-Abu-(CO)- GMSYS-Am | 130 |
| 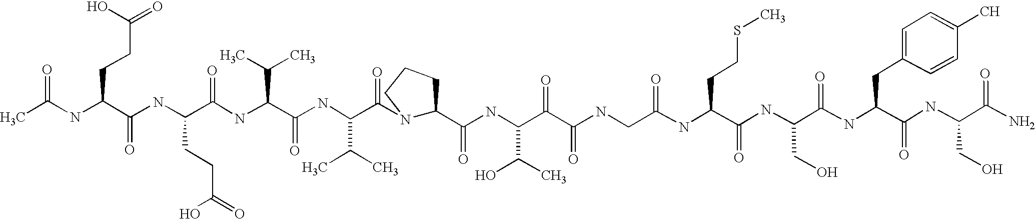 | Ac-EEVVP-(s,s)alloT-(CO)- GMSYS-Am | 60 |
| 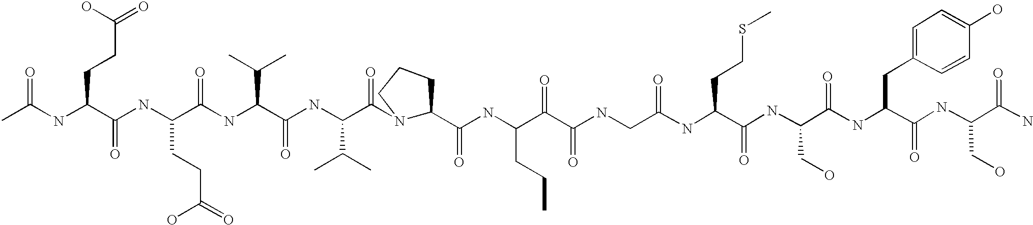 | Ac-EEVVP-G(propynyl)- (CO)-GMSYS-Am | 9 |

What is claimed is:

1. A compound, including enantiomers, stereoisomers, rotomers and tautomers of said compound, and pharmaceutically acceptable salts, solvates or derivatives thereof, with said compound having the general structure shown in Formula I: 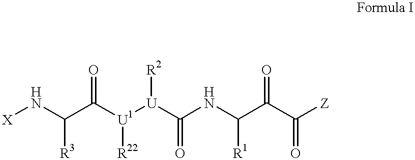
or a pharmaceutically acceptable derivative thereof, where X is: COCH(R⁴)NHCO—CH(R⁵)NHCOCH(R⁶)NHCORⁿ or COCH(R⁴)NHCOCH(R⁵)NHCOCH(R⁶)—NHSO₂R²⁰;
U¹ is a nitrogen atom and U is —CH—;
Z is: NH—CH(R^{1′})CONHCH(R^{2′})CONHCH(R^{3′})CONHCH(R^{4′})CONHCH(R^{5′})COR^{c};
R¹, R², R²², R³, R⁴, R⁵, R⁶, Rⁿ, R^{2′}, R^{3′}, R^{4′}, R^{5′}, R^{1′} R²⁰, and R^{c} are selected from (a) and (b) as follows:
(a) R¹ is selected from (i)-(v) as follows:
(i) C₁₋₂ alkyl substituted with Q;
(ii) C₃₋₁₀ alkyl that is unsubstituted or substituted with Q;
(iii) cycloalkyl that is unsubstituted or substituted with Q;
(iv) alkenyl that is unsubstituted or substituted with Q; or
(v) alkynyl that is unsubstituted or substituted with Q;
R² and R²² are selected from (i) or (ii) as follows:
(i) R² and R²² together form alkylene, alkenylene, thiaalkylene, thiaalkenylene, alkylenethiaalkylene, alkyleneazaalkylene, arylene, alkylenearylene or dialkylenearylene; or
(ii) R² and R²² are each independently selected from H, alkyl, cycloalkyl, aralkyl and heteroaralkyl;
R³ is selected from the group consisting of alkyl, cycloalkyl, aryl, aralkyl, heteroaryl and heteroaralkyl;
R⁴ is alkyl, cycloalkyl, heteroaralkyl or aralkyl;
R⁵ is alkyl or cycloalkyl;
R⁶ is alkyl or cycloalkyl;
Rⁿ is alkyl, alkenyl, alkynyl, alkoxy, aryl, aralkyl, aralkenyl, aralkynyl, aryloxy, aralkoxy, heteroaryl, heteroaralkyl, heteroaralkenyl, heteroaralkynyl, heteroaryloxy, heteroaralkoxy or NR³⁰R³¹;
R³⁰ and R³¹ are each independently selected from the group consisting of H, alkyl, aryl, heteroaryl, aralkyl and heteroaralkyl;
R^{2′} is H, alkyl, cycloalkyl, aryl, heteroaryl, aralkyl or heteroaralkyl;
R^{3′} is selected from the group consisting of alkyl, cycloalkyl, aralkyl and heteroaralkyl;
R^{4′} is aralkyl or heteroaralkyl;
R^{5′} is alkyl or cycloalkyl;
R^{1′} is selected from H, alkyl, cycloalkyl, aralkyl and heteroaralkyl;
R²⁰ is alkyl, alkenyl, alkynyl, aryl, aralkyl, aralkenyl, aralkynyl, heteroaryl, heteroaralkyl, heteroaralkenyl or heteroaralkynyl;
R^{c} is selected from amino, hydroxy, alkoxy, cycloalkoxy, alkylamino, alkenyloxy, alkenylamino, aryloxy, heteroaryloxy, arylamino, heteroarylamino, aralkoxy, heteroaralkoxy, aralkylamino and heteroaralkylamino;
Q is halide, pseudohalide, hydroxy, nitrile, formyl, mercapto, alkyl, haloalkyl, polyhaloalkyl, alkenyl containing 1 double bond, alkynyl containing 1 triple bond, cycloalkyl, cycloalkylalkyl, alkylidene, alkylcarbonyl, alkoxy, perfluoroalkoxy, alkylcarbonyloxy or alkylthio; and
R², R²², R³, R⁴, R⁵, R⁶, Rⁿ, R^{2′}, R^{3′}, R^{4′}, R^{5′}, R^{1′}, R²⁰, and R^{c} are unsubstituted or substituted with one or more substituents each independently selected from Q¹, where Q¹ is halide, pseudohalide, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, ureido, alkylureido, arylureido, amino, aminoalkyl, alkylaminoalkyl, dialkylaminoalkyl, arylaminoalkyl, diarylaminoalkyl, alkylarylaminoalkyl, alkylamino, dialkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonyl- amino, azido, dialkylphosphonyl, alkylarylphosphonyl, diarylphosphonyl, alkylthio, arylthio, perfluoroalkylthio, hydroxycarbonylalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl, arylaminosulfonyl, diarylaminosulfonyl or alkylarylaminosulfonyl; and
the aryl and heteroaryl groups of Q¹ are unsubstituted or substituted with one or more substituents each independently selected from Q², where Q² is alkyl, halide, pseudohalide, alkoxy, aryloxy or alkylenedioxy; or
(b) R¹ and R³, and/or R² and R⁴, and/or R³ and R⁵, and/or R⁴ and R⁶, and/or R¹ and R^{2′}, and/or R^{1′} and R^{3′}, and/or R^{2′} and R^{4′}, and/or R^{3′} and R^{5′}, and/or R² and R^{1′}, and/or R¹ and R^{1′} together form alkylene, alkenylene, alkylenearylene, dialkylenearylene, alkylene-OC(O)-alkylene, alkylene-NHC(O)-alkylene, alkylene-O-alkylene, alkylene-NHC(O)-alkylene-NHC(O)-alkylene, alkylene-C(O)NH-alkylene-NHC(O)-alkylene, alkylene-NHC(O)-alkylene-C(O)NH-alkylene, alkylene-S(O)m-S(O)m-alkylene or alkylene-S(O)ₘ-alkylene where m is 0-2, and the alkylene and arylene portions are unsubstituted or substituted with Q¹; and the others are chosen as in (a).

2. The compound of claim 1, wherein Z is:
NH—CH(R^{1′})CONHCH(R^{2′})CONHCH(R^{3′})CONHCH(R^{4′})CONHCH(R^{5′})COR^{c},:
and R¹ is selected from (i)-(iv) as follows:
(i) C₁₋₂ alkyl that is substituted with Q;
(ii) C₃₋₁₀ alkyl that is unsubstituted or substituted with Q;
(iii) alkenyl that is unsubstituted or substituted with Q; or
(iv) alkynyl that is unsubstituted or substituted with Q;
R² and R²² are selected from (i) or (ii) as follows:
(i) R² and R²² together form alkylene, thiaalkylene, or dialkylenearylene; or
(ii) R² and R²² are each independently selected from H, alkyl and aralkyl;
R³ is selected from the group consisting of alkyl, cycloalkyl, aryl and aralkyl;
R⁴ is alkyl, heteroaralkyl or aralkyl;
R⁵ is alkyl;
R⁶ is alkyl;
Rⁿ is alkyl, hydroxycarbonylalkyl, alkoxy, heteroaryl, aryl or aralkyl;
R^{2′} is H, alkyl, cycloalkyl, aryl or aralkyl;
R^{3′} is selected from the group consisting of alkyl and heteroaralkyl;
R^{4′} is aralkyl;
R^{5′} is alkyl;
R^{1′} is selected from H, alkyl and aralkyl;
R²⁰ is alkyl, aryl, aralkyl or aralkenyl;
R^{c} is selected from amino, hydroxy, alkoxy, alkenyloxy, alkylamino, alkenylamino and aralkylamino;
Q is halide, pseudohalide, hydroxy, nitrile, formyl, mercapto, alkyl, haloalkyl, polyhaloalkyl, alkenyl containing 1 double bond, alkynyl containing 1 triple bond, cycloalkyl, cycloalkylalkyl, alkylidene, alkylcarbonyl, alkoxy, perfluoroalkoxy, alkylcarbonyloxy or alkylthio; and
R² R²², R³, R⁴, R⁵, R⁶, Rⁿ, R^{2′}, R^{3′}, R^{4′}, R^{5′}, R^{1′}, R²⁰, and R^{c} are unsubstituted or substituted with one or more substituents each independently selected from Q¹, where Q¹ is halide, pseudohalide, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, ureido, alkylureido, arylureido, amino, aminoalkyl, alkylaminoalkyl, dialkylaminoalkyl, arylaminoalkyl, diarylaminoalkyl, alkylarylaminoalkyl, alkylamino, dialkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, azido, dialkylphosphonyl, alkylarylphosphonyl, diarylphosphonyl, alkylthio, arylthio, perfluoroalkylthio, hydroxycarbonylalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl, arylaminosulfonyl, diarylaminosulfonyl or alkylarylaminosulfonyl; and
the aryl and heteroaryl groups of Q¹ are unsubstituted or substituted with one or more substituents each independently selected from Q², where Q² is alkyl, halide, pseudohalide, alkoxy, aryloxy or alkylenedioxy.

3. The compound of claim 2, wherein:
R¹ is C₃₋₁₀ alkyl, or is alkenyl or alkynyl, and is unsubstituted or substituted with Q;
R² and R²² are selected from (i) or (ii) as follows:
(i) R² and R²² together form propylene, butylene or 1,2-dimethylenephenylene, where the butylene and 1,2-dimethylenephenylene groups are unsubstituted and the propylene group is unsubstituted or is substituted with 4-methoxyphenylsulfonylamino, N-phenylureidomethyl, methyl, benzoylaminomethyl, phenyl, 3-phenoxybenzoylaminomethyl, N-phenylureido, phenylsulfonylaminomethyl, 9-fluorenylmethoxy-carbonylaminomethyl, phenoxycarbonylaminomethyl, iso-butoxy-carbonylamino, hydroxycarbonylmethyl, hydroxycarbonylmethoxy, 2-propen-1-yl, N-(4-methoxyphenyl)ureido, 3-phenoxybenzoylamino, 4-methoxyphenylmethyl, 9-fluorenylmethoxycarbonylamino, benzyl, 4-methoxybenzoylamino, benzoylamino, 3,4-methylenedioxybenzoylamino, 4-fluorobenzoylamino, phenylsulfonylamino, 4-phenoxybenzoylamino or amino; or
(ii) R² is selected from CH₂SO₂Me, CH₂SCH₂COOH, CH₂CH₂COOH and CH₂SMe; and R²² is H; and
R³ is i-Pr, cyclohexyl or 1-methyl-1-propyl.

4. The compound of claim 2, wherein:
R¹ is C₃₋₁₀ alkyl, or is alkenyl or alkynyl, and is unsubstituted or substituted with Q;
R² and R²² are selected from (i) or (ii) as follows:
(i) R² and R²² together form propylene or 1,2-dimethylenephenylene, where the 1,2-dimethylenephenylene group is unsubstituted and the propylene group is unsubstituted or is substituted with 4-methoxyphenylsulfonylamino, N-phenylureidomethyl, methyl, benzoylaminomethyl, phenyl, 3-phenoxybenzoylaminomethyl, N-phenylureido, phenylsulfonylaminomethyl, 9-fluorenylmethoxy-carbonylaminomethyl, phenoxycarbonylaminomethyl, iso-butoxy-carbonylamino, hydroxycarbonylmethyl or hydroxycarbonylmethoxy; or
(ii) R² is selected from CH₂SO₂Me and CH₂SCH₂COOH; and R²² is H; and
R³ is i-Pr, cyclohexyl or 1-methyl-1-propyl.

5. The compound of claim 2, wherein:
R¹ is unsubstituted C₃₋₁₀ alkyl;
R² and R²² together form propylene or 1,2-dimethylenephenylene, where the 1,2-dimethylenephenylene group is unsubstituted and the propylene group is unsubstituted or is substituted with 4-methoxyphenylsulfonylamino, N-phenyl-ureidomethyl, methyl, benzoylaminomethyl, phenyl, 3-phenoxybenzoylaminomethyl, N-phenylureido, phenylsulfonylaminomethyl, 9-fluorenyl-methoxycarbonylaminomethyl, phenoxycarbonylaminomethyl, iso-butoxy-carbonylamino, hydroxycarbonylmethyl or hydroxycarbonylmethoxy; and
R³ is i-Pr, cyclohexyl or 1-methyl-1-propyl.

6. The compound of claim 6, wherein R¹ is n-Pr; and R² and R²² together form unsubstituted propylene.

7. The compound of claim 1, wherein X is:
COCH(R⁴)NHCOCH(R⁵)NHCOCH(R⁶)NHCORⁿ.

8. The compound of claim 7, wherein:
R¹ is C₃₋₁₀ alkyl, or is alkenyl or alkynyl, and is unsubstituted or substituted with Q;
R² and R²² are selected from (i) or (ii) as follows:
(i) R² and R²² together form propylene, butylene or 1,2-dimethylenephenylene, where the butylene and 1,2-dimethylenephenylene groups are unsubstituted and the propylene group is unsubstituted or is substituted with 4-methoxyphenylsulfonylamino, N-phenylureidomethyl, methyl, benzoylaminomethyl, phenyl, 3-phenoxybenzoylaminomethyl, N-phenylureido, phenylsulfonylaminomethyl, 9-fluorenylmethoxy-carbonylaminomethyl, phenoxycarbonylaminomethyl, iso-butoxy-carbonylamino, hydroxycarbonylmethyl, hydroxycarbonylmethoxy, 2-propen-1-yl, N-(4-methoxyphenyl)ureido, 3-phenoxybenzoylamino, 4-methoxyphenylmethyl, 9-fluorenylmethoxycarbonylamino, benzyl, 4-methoxybenzoylamino, benzoylamino, 3,4-methylenedioxybenzoylamino, 4-fluorobenzoylamino, phenylsulfonylamino, 4-phenoxybenzoylamino or amino; or
(ii) R² is selected from CH₂SO₂Me, CH₂SCH₂COOH, CH₂CH₂COOH and CH₂SMe; and R²² is H; and
R³ is i-Pr, cyclohexyl or 1-methyl-1-propyl.

9. The compound of claim 7, wherein:
R¹ is C₃₋₁₀ alkyl, or is alkenyl or alkynyl, and is unsubstituted or substituted with Q;
R² and R²² are selected from (i) or (ii) as follows:
(i) R² and R²² together form propylene or 1,2-dimethylenephenylene, where the 1 ,2-dimethylenephenylene group is unsubstituted and the propylene group is unsubstituted or is substituted with 4-methoxyphenylsulfonylamino, N-phenylureidomethyl, methyl, benzoylaminomethyl, phenyl, 3-phenoxybenzoylaminomethyl, N-phenylureido, phenylsulfonylaminomethyl, 9-fluorenylmethoxy-carbonylaminomethyl, phenoxycarbonylaminomethyl, iso-butoxy-carbonylamino, hydroxycarbonylmethyl or hydroxycarbonylmethoxy; or
(ii) R² is selected from CH₂SO₂Me and CH₂SCH₂COOH; and R²² is H; and
R³ is i-Pr, cyclohexyl or 1-methyl-1-propyl.

10. The compound of claim 9, wherein:
R¹ is unsubstituted C₃₋₁₀ alkyl;
R² and R²² together form propylene or 1,2-dimethylenephenylene, where the 1,2-dimethylenephenylene group is unsubstituted and the propylene group is unsubstituted or is substituted with 4-methoxyphenylsulfonylamino, N-phenyl-ureidomethyl, methyl, benzoylaminomethyl, phenyl, 3-phenoxybenzoylaminomethyl, N-phenylureido, phenylsulfonylaminomethyl, 9-fluorenyl-methoxycarbonylaminomethyl, phenoxycarbonylaminomethyl, iso-butoxy-carbonylamino, hydroxycarbonylmethyl or hydroxycarbonylmethoxy; and
R³ is i-Pr, cyclohexyl or 1-methyl-1-propyl.

11. The compound of claim 10, wherein R¹ is n-Pr; and R² and R²² together form unsubstituted propylene.

12. The compound of claim 7, wherein:
R⁴ is alkyl, heteroaralkyl or aralkyl;
R⁵ is alkyl;
R⁶ is alkyl; and
Rⁿ is alkyl, alkoxy, heteroaryl, aryl or aralkyl.

13. The compound of claim 7, wherein:
R⁴ is i-Pr;
R⁵ and R⁶ are CH₂CH₂COOH; and
Rⁿ is methyl.

14. The compound of claim 2, wherein:
R^{2′} is CH₂CH₂SMe, C(OH)Me, CH₂CH₂S(O)Me, phenyl or CH₂C(O)NH₂;
R^{3′} is hydroxymethyl, hydroxycarbonylmethyl or 4-imidazolylmethyl;
R^{4′} is 4-hydroxyphenylmethyl;
R^{5′} is hydroxymethyl; and
R^{1′} is H.

15. The compound of claim 6, wherein:
R^{2′} is H, alkyl or aryl;
R^{3′} is alkyl or heteroaralkyl;
R^{4′} is aralkyl;
R^{5′} is alkyl; and
R^{1′} is H, alkyl or aralkyl.

16. The compound of claim 6, wherein:
R^{2′} is CH₂CH₂SMe, C(OH)Me, CH₂CH₂S(O)Me, phenyl or CH₂C(O)NH₂;
R^{3′} is hydroxymethyl, hydroxycarbonylmethyl or 4-imidazolylmethyl;
R^{4′} is 4-hydroxyphenylmethyl;
R^{5′} is hydroxymethyl; and
R^{1′} is H.

17. The compound of claim 1, wherein the compound is selected from the group consisting of:
AcEEVVPnV-(CO)-GMSYS-Am
AcEEVVPnV-CO-GMdSYS-Am
AcEEVVPnV-CO-GMdHYS-Am
AcEEVVPnV-CO-GMdDYS-Am
AcEEVVPnV-CO-GdMSYS-Am
AcEEVVPnV-CO-GdMdSYS-Am
AcEEVVPnV-CO-GdMHYS-Am
AcEEVVPnV-CO-GdMDYS-Am
AcEEVVPnV-CO-GdMdDYS-Am
AcEEVVPnV-CO-GGSYS-Am
AcEEVVPnV-CO-GGHYS-Am
AcEEVVPnV-CO-GGdHYS-Am
AcEEVVPnV-CO-GGDYS-Am
AcEEVVPnV-CO-GGdDYS-Am
AcEEVVPnV-CO-GQSYS-Am
AcEEVVPnV-CO-GQdSYS-Am
AcEEVVPnV-CO-GQd HYS-Am
AcEEVVPnV-CO-GQdDYS-Am
AcEEVVPnV-CO-GdQSYS-Am
AcEEVVPnV-CO-GdQdSYS-Am
AcEEVVPnV-CO-GdQHYS-Am
AcEEVVPnV-CO-GdQDYS-Am
AcEEVVPnV-CO-GdQdDYS-Am
AcEEVVPnV-CO-GTSYS-Am
AcEEVVPnV-CO-GTdSYS-Am
AcEEVVPnV-CO-GTHYS-Am
AcEEVVPnV-CO-GTDYS-Am
AcEEVVPnV-CO-GTdDYS-Am
AcEEVVPnV-CO-GSdSYS-Am
AcEEVVPnV-CO-GSdHYS-Am
AcEEVVPnV-CO-GSdDYS-Am
AcEEVVPnV-CO-GdSSYS-Am
AcEEVVPnV-CO-GdSdSYS-Am
AcEEVVPnV-CO-GdSHYS-Am
AcEEVVPnV-CO-GdSdHYS-Am
AcEEVVPnV-CO-GdSDYS-Am
AcEEVVPnV-CO-GdSdDYS-Am
AcEEVVPnV-CO-GM(O)HYS-Am
AcEEVVPnV-(CO)-GdM(O)SYS-Am
AcEEVVPnV-CO-GdM(O)dHYS-Am
AcEEVVPnV-CO-GdM(O)DYS-Am
AcEEVVPnV-CO-GdM(O)dDYS-Am
Ac-EEVVP-V-(CO)-GMSYS-Am
Ac-EEVVP-L-(CO)-GMSYS-Am
Ac-EEVVP-nL-(CO)-GMSYS-Am
Ac-EEVVP-Abu-(CO)-GMSYS-Am
Ac-EEVVP-(s,s)alloT-(CO)-GMSYS-Am
Ac-EEVVP-G(propynyl)-(CO)-GMSYS-Am

18. The compound of claim 1, wherein the compound is selected from the group consisting of:
AcEEVVPnV-CO-GdMDYS-Am
AcEEVVPnV-CO-GdMdDYS-Am
AcEEVVPnV-CO-GGSYS-Am
AcEEVVPnV-CO-GGHYS-Am
AcEEVVPnV-CO-GGDYS-Am
AcEEVVPnV-CO-GGdDYS-Am
AcEEVVPnV-CO-GQSYS-Am
AcEEVVPnV-CO-GQdSYS-Am
AcEEVVPnV-CO-GQdHYS-Am
AcEEVVPnV-CO-GQdDYS-Am
AcEEVVPnV-CO-GdQSYS-Am
AcEEVVPnV-CO-GdQdSYS-Am
AcEEVVPnV-CO-GdQHYS-Am
AcEEVVPnV-CO-GdQDYS-Am
AcEEVVPnV-CO-GdQdDYS-Am
AcEEVVPnV-CO-GTSYS-Am
AcEEVVPnV-CO-GTdSYS-Am
AcEEVVPnV-CO-GTHYS-Am
AcEEVVPnV-CO-GTDYS-Am
AcEEVVPnV-CO-GTd DYS-Am
AcEEVVPnV-CO-GSdSYS-Am
AcEEVVPnV-CO-GSdHYS-Am
AcEEVVPnV-CO-GSdDYS-Am
AcEEVVPnV-CO-GdSSYS-Am
AcEEVVPnV-CO-GdSdSYS-Am
AcEEVVPnV-CO-GdSHYS-Am
AcEEVVPnV-CO-GdSdHYS-Am
AcEEVVPnV-CO-GdSDYS-Am
AcEEVVPnV-CO-GdSdDYS-Am
AcEEVVPnV-CO-GM(O)HYS-Am
AcEEVVPnV-(CO)-GdM(O)SYS-Am
AcEEVVPnV-CO-GdM(O)DYS-Am
AcEEVVPnV-CO-GdM(O)dDYS-Am
Ac-EEVVP-(s,s)alloT-(CO)-GMSYS-Am
Ac-EEVVP-G(propynyl)-(CO)-GMSYS-Am

19. A pharmaceutical composition comprising as an active ingredient a compound of claim 1.

20. The pharmaceutical composition of claim 19 for use in treating disorders associated with Hepatitis C virus.

21. The pharmaceutical composition of claim 19 additionally comprising a pharmaceutically acceptable carrier.

22. The pharmaceutical composition of claim 21, additionally containing an antiviral agent.

23. The pharmaceutical composition of claim 22, still additionally containing an interferon.

24. The pharmaceutical composition of claim 23, wherein said antiviral agent is ribavirin and said interferon is α-interferon.

25. A method of treating disorders associated with the HCV protease, said method comprising administering to a patient in need of such treatment a pharmaceutical composition which composition comprises therapeutically effective amounts of a compound of claim 1.

26. The method of claim 25, wherein said administration is subcutaneous.

27. The use of a compound of claim 1 for the manufacture of a medicament to treat disorders associated with the HCV protease.

28. A method of preparing a pharmaceutical composition for treating disorders associated with the HCV protease, said method comprising bringing into intimate contact a compound of claim 1 and a pharmaceutically acceptable carrier.

29. A compound exhibiting HCV protease inhibitory activity, including enantiomers, stereoisomers, rotamers and tautomers of said compound, and pharmaceutically acceptable salts or solvates of said compound, said compound being selected from the group of compounds in claim 17.

30. A pharmaceutical composition for treating disorders associated with the HCV protease, said composition comprising therapeutically effective amount of one or more compounds in claim 17 and a pharmaceutically acceptable carrier.
